(19) United States
(12) Patent Application Publication
(10) Pub. No.: US 2025/0144031 A1
(43) Pub. Date: May 8, 2025

(54) COMPOSITION FOR TREATING SPINAL MUSCULAR ATROPHY

(71) Applicant: Hoffmann-La Roche Inc., Little Falls, NJ (US)

(72) Inventors: Reto MAURER, Basel (CH); Serge OBERDORF, Blotzheim (FR); Stefanie PETER, Luzern (CH); Katharina SENGER, Sitzenkirch (DE)

(73) Assignee: Hoffmann-La Roche Inc., Little Falls, NJ (US)

(21) Appl. No.: 18/926,622

(22) Filed: Oct. 25, 2024

(30) Foreign Application Priority Data
Oct. 27, 2023 (EP) 23206351.1

(51) Int. Cl. A61K 9/28; A61K 9/20; A61K 31/519

(52) U.S. Cl. CPC A61K 9/2853; A61K 9/2009; A61K 9/2018; A61K 9/2027; A61K 9/2054; A61K 9/2813; A61K 9/2886; A61K 31/519

(57) ABSTRACT

Disclosed herein are pharmaceutical compositions and dosage forms including risdiplam that are useful in the treatment of subjects having SMA. The present disclosure also provides methods for preparing these pharmaceutical compositions and dosage forms, and methods of treating subjects having SMA utilizing the pharmaceutical compositions and dosage forms provided herein.

### CROSS REFERENCE TO RELATED APPLICATIONS

This application claims priority to EP application number 23206351.1 filed Oct. 27, 2023, which is incorporated herein by reference in its entirety.

The present invention provides pharmaceutical formulation comprising a compound which is SMN2 gene splicing modulator, in particular wherein the SMN2 gene splicing modulator is risdiplam, their manufacture and their use for the treatment, delay of progression or amelioration of Spinal muscular atrophy (SMA). The present disclosure also provides methods for preparing these pharmaceutical formulation and dosage forms, and methods of treating, delaying of progression or ameliorating of SMA utilizing the pharmaceutical formulation and dosage forms provided herein. The present invention is generally directed to a patient-easier drug delivery system for targeted populations. Specifically, the present invention relates to a pharmaceutical composition in the form of a tablet, more particularly a film-coated tablet. In particular, the present invention provides compliant dosage forms especially for patients who have a body weight of more than or equal to 20 kg. The tablet can be conventionally be swallowed as a whole tablet or be dispersed in a small amount of water prior administration, in order to provide a liquid form, especially in case the patient has difficulty to swallow or for the pediatric population.

In particular, the present invention relates to a pharmaceutical composition comprising: (a) a compound of formula (I)

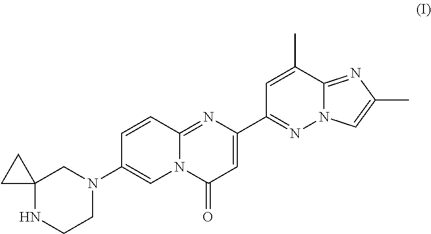 [SMILES: Cc1cn2nc(-c3cc(=O)n4cc(N5CCNC6(CC6)C5)ccc4n3)cc(C)c2n1]

or a pharmaceutically acceptable salt thereof;
(b) crospovidone; and
(c) an acidifier selected from tartaric acid, citric acid or malic acid, more particularly tartaric acid.

Spinal muscular atrophy (SMA), in its broadest sense, describes a collection of inherited and acquired central nervous system (CNS) diseases characterized by progressive motor neuron loss in the spinal cord and brainstem causing muscle weakness and muscle atrophy. SMA is characterized by a degeneration of the alpha motor neurons from the anterior horn of the spinal cord leading to muscular atrophy and resulting in paralysis. This alpha motor neuron degeneration thus substantially compromises the vital prognosis of patients. In healthy subjects, these neurons transmit messages from the brain to the muscles, leading to the contraction of the latter. In the absence of such a stimulation, the muscles atrophy. Subsequently, in addition to a generalized weakness and atrophy of the muscles, and more particularly of those of the trunk, upper arms and thighs, these disorders can be accompanied by serious respiratory problems.

Infantile SMA is the most severe form of this neurodegenerative disorder. Symptoms include muscle weakness, poor muscle tone, weak cry, limpness or a tendency to flop, difficulty sucking or swallowing, accumulation of secretions in the lungs or throat, feeding difficulties, and increased susceptibility to respiratory tract infections. The legs tend to be weaker than the arms and developmental milestones, such as lifting the head or sitting up, cannot be reached. In general, the earlier the symptoms appear, the shorter the lifespan. As the motor neuron cells deteriorate, symptoms appear shortly afterward. The severe forms of the disease are fatal and all forms have no known cure. The course of SMA is directly related to the rate of motor neuron cell deterioration and the resulting severity of weakness. Infants with a severe form of SMA frequently succumb to respiratory disease due to weakness in the muscles that support breathing. Children with milder forms of SMA live much longer, although they may need extensive medical support, especially those at the more severe end of the spectrum. The clinical spectrum of SMA disorders has been divided into the following five groups:

1. Type 0 SMA (In Utero SMA) is the most severe form of the disease and begins before birth. Usually, the first symptom of Type 0 SMA is reduced movement of the fetus that can first be observed between 30 and 36 weeks of pregnancy. After birth, these newborns have little movement and have difficulties with swallowing and breathing.

2. Type I SMA (Infantile SMA or Werdnig-Hoffmann disease) presents symptoms between 0 and 6 months; this form of SMA is very severe. Patients never achieve the ability to sit, and death usually occurs within the first 2 years without ventilatory support.

3. Type II SMA (Intermediate SMA) has an age of onset at 7-18 months. Patients achieve the ability to sit unsupported, but never stand or walk unaided. Prognosis in this group is largely dependent on the degree of respiratory involvement.

4. Type III SMA (Juvenile SMA or Kugelberg-Welander disease) is generally diagnosed after 18 months. Type 3 SMA individuals are able to walk independently at some point during their disease course but often become wheelchair-bound during youth or adulthood.

5. Type IV SMA (Adult onset SMA). Weakness usually begins in late adolescence in the tongue, hands, or feet, then progresses to other areas of the body. The course of adult SMA is much slower and has little or no impact on life expectancy.

All the forms of Spinal muscular atrophy are accompanied by progressive muscle weakness and atrophy subsequent to the degeneration of the neurons from the anterior horn of the spinal cord. SMA currently constitutes one of the most common causes of infant mortality. It equally affects girls or boys in all regions of the world with a prevalence of between 1/6000 and 1/10000.

The goal of any drug delivery system is to provide a therapeutic amount of drug to the proper site in the body to achieve and then maintain the desired drug concentration. Risdiplam was first approved as a 0.75 mg/ml powder for oral solution due to the patient's profile. The powder for Oral Solution (PfOS) formulation, the first approved formulation on the market, was developed to enable flexible dosing for all patients based on their body weight going from newborns to adults. Due to the spectrum of SMA as outlined above, treatment options for patients at birth through adulthood are necessary, and the ability to accurately dose across such a broad range of patient weights is critical, especially for patients with greater than 20 kg. Due to special requirements for reconstitution, the reconstitution of the PfOS has to be performed in a hospital pharmacy and the bottle requires refrigerated storage afterwards, which is inconvenient for patients and requires a complex supply chain.

The powder for oral solution, especially when reconstituted in solution, may face some stability burdens under some specific conditions. To ensure product quality over the intended application time, the reconstituted PfOS requires refrigerated storage. Notably, the present invention addresses those limitations.

The formulation composition of the film-coated tablet was selected in order to provide a stable conventional film-coated tablet, which does not require a refrigerated storage.

In addition, the composition contains tartaric acid, as an acidifier, which ensures complete dissolution of Risdiplam also at elevated and variable stomach pH. It was found that Risdiplam's solubility is influenced by the pH of the solution. Therefore by lowering the pH with tartaric acid, risdiplam can dissolve completely. Bioequivalency of the tablet in comparison to the PfOS was confirmed. Bioequivalency in the fed and fasted state was shown for both, the tablet swallowed as whole but also dispersed in a small amount of water.

According to the present invention the core tablet formulation disintegrates surprisingly fast. The presence of Crospovidone is responsible for this fast disintegration. Thus, the film-coated tablet can be dispersed in water in order to enable the use by patients who have difficulty to swallow the tablets.

It is common to coat tablets with a film-coating. Standard coatings for a film-coated tablet are usually used to reduce dust, handling with care, esthetic features and to distinguish between dose strength. Iron-oxides are widely used as colorants, in order to maintain a homogenous appearance. It was surprisingly found that risdiplam is chemically incompatible with iron oxides. As described in further detail in the Examples, the presence of iron oxide led to significant and serious degradation of Risdiplam, and extreme discoloration of the tablet. Therefore, the present invention had to ensure that the tablet is not in direct contact with an iron-oxide coating. Therefore the present invention comprises the use of a first coating which is free of iron oxide, preventing the interaction of risdiplam with the second coating which may comprise Iron-oxides for color-determination such as a yellow coating.

### BRIEF DESCRIPTION OF THE FIGURES

FIG. 1: Dissolution Performance at pH 4.5 of Risdiplam in the presence of acidifier, Film-Coated Tablets, 5 mg, Containing Different Levels of Tartaric Acid (TA), Formulation 1 comprises 0.00 mg of TA, Formulation 2 comprises 2.00 mg of TA, Formulation 3 comprises 3.00 mg of TA, Formulation 4 comprises 4.00 mg of TA, Formulation 5.00 comprises 6.00 mg of TA, Formulation 6 comprises 6.0000 mg of TA, Formulation 7 comprises 7.53 mg of TA.

FIG. 2: Risdiplam Tablets

FIG. 3: Discoloration and decomposition of risdiplam in the presence of Sucralose, a) Risdiplam+Sucralose at time 0, b) risdiplam+Sucralose (70° C./75% Relative Humidity) after 15 days, c) sucralose without Risdiplam (70° C./75% Relative Humidity) after 15 days.

FIG. 4: Formulation 12 without coating openly for two weeks at 70° C./75% Relative Humidity and at 70° C./5% Relative Humidity.

FIG. 5: a) Placebo tablet after two weeks at 70° C./5% Relative Humidity, b) Placebo tablet after two weeks at 70° C./75% Relative Humidity.

FIG. 6: a) tablet of Formulation 14 at time t0, b) tablet of Formulation 14 after two weeks at 70° C./5% Relative Humidity, c) tablet of Formulation 14 after two weeks at 70° C./75% Relative Humidity, d) tablet of Formulation 15 at time t0, e) tablet of Formulation 15 after two weeks at 70° C./5% Relative Humidity, f) tablet of Formulation 15 after two weeks at 70° C./75% Relative Humidity.

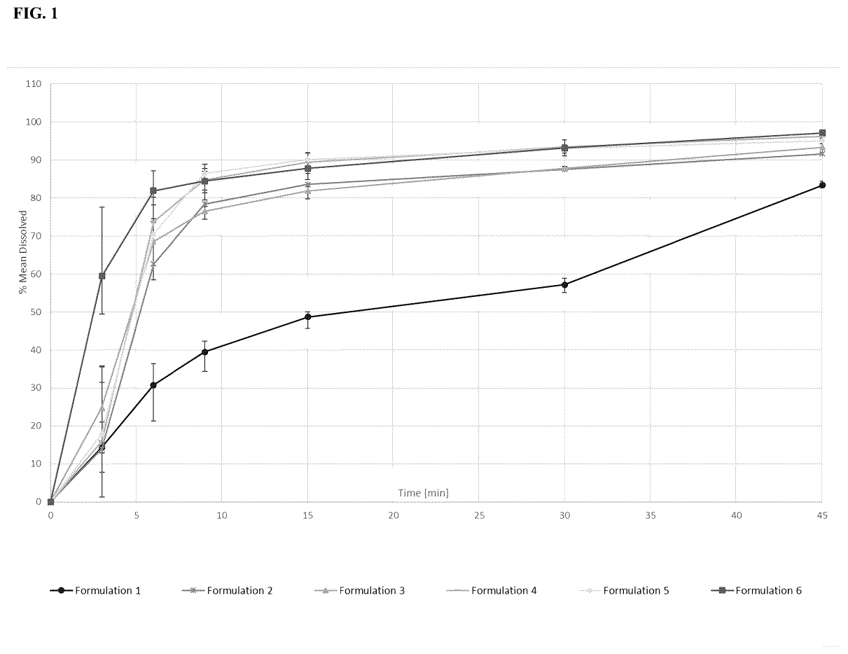

All publications, patent applications, patents and other references mentioned herein are incorporated by reference in their entirety.

The nomenclature used in the present application is based on IUPAC systematic nomenclature, unless indicated otherwise.

Various features and embodiments of the present invention are disclosed herein, however other features of the invention, modifications and equivalents will be apparent to a person skilled in the relevant art, based on the teachings provided. The invention described is not limited to the examples and embodiments provided, various alternatives equivalents will be appreciated by those skilled in the art. As used herein, the singular forms “a”, “an” and “the” include the plural unless the context clearly dictates otherwise. For example, “a” individual will also include “individuals”.

Unless otherwise stated, the following terms used in the specification and claims have the meanings given below:

“about” or “approximately” refers to a range values that fall within 5%, greater or less than the stated reference value.

“Individual” or “subject” used interchangeably, is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In a particular embodiment of the invention the subject is a human with Spinal muscular atrophy (SMA). In another specific embodiment, the subject is a human with SMA caused by an inactivating mutation or deletion in the SMN1 gene on both chromosomes, resulting in a loss of SMN1 gene function.

“Spinal muscular atrophy” (or SMA) refers to a disease caused by an inactivating mutation or deletion in the SMN1 gene on both chromosomes, resulting in a loss of SMN1 gene function. Symptoms of SMA—depending on the type of SMA—include muscle weakness, poor muscle tone, weak cry, weak cough, limpness or a tendency to flop, difficulty sucking or swallowing, difficulty breathing, accumulation of secretions in the lungs or throat, clenched fists with sweaty hand, flickering/vibrating of the tongue, head often tilted to one side, even when lying down, legs that tend to be weaker than the arms, legs frequently assuming a “frog legs” position, feeding difficulties, increased susceptibility to respiratory tract infections, bowel/bladder weakness, lower-than-normal weight, inability to sit without support, failure to walk, failure to crawl, and hypotonia, areflexia, and multiple congenital contractures (arthrogryposis) associated with loss of anterior horn cells.

“Treating Spinal muscular atrophy (SMA)” or “treatment of Spinal muscular atrophy (SMA)” refers to one or more of the following effects: (i) reduction or amelioration of the severity of SMA; (ii) delay of the onset of SMA; (iii) inhibition of the progression of SMA; (iv) reduction of hospitalization of a subject; (v) reduction of hospitalization length for a subject; (vi) increase of the survival of a subject; (vii) improvement of the quality of life of a subject; (viii) reduction of the number of symptoms associated with SMA; (ix) reduction of or amelioration of the severity of one or more symptoms associated with SMA; (x) reduction of the duration of a symptom associated with SMA; (xi) prevention of the recurrence of a symptom associated with SMA; (xii) inhibition of the development or onset of a symptom of SMA; and/or (xiii) inhibition of the progression of a symptom associated with SMA. More particular, “treating SMA” denotes one or more of the following beneficial effects: (i) a reduction in the loss of muscle strength; (ii) an increase in muscle strength; (iii) a reduction in muscle atrophy; (iv) a reduction in the loss of motor function; (v) an increase in motor neurons; (vii) a reduction in the loss of motor neurons; (viii) protection of SMN deficient motor neurons from degeneration; (ix) an increase in motor function; (x) an increase in pulmonary function; and/or (xi) a reduction in the loss of pulmonary function. More particularly, “Treating SMA” results in the functional ability or helps retain the functional ability for a human child or human adult to sit up unaided or for a human child or a human adult to stand up unaided, to walk unaided, to run unaided, to breathe unaided, to turn during sleep unaided, or to swallow unaided.

“Patient” or “patients” refers to a human (such as a male or female human) who has been diagnosed with SMA.

“Active pharmaceutical ingredient” (or “API”) refers to the compound or molecule in a pharmaceutical composition that has a particular biological activity. More particularly “API” refers to the active substance which is according to the invention, risdiplam.

“Pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffer system, tonicity agents, stabilizers, antioxidants, surfactants, carriers, diluents or lubricants used in formulating pharmaceutical products.

“Pharmaceutical composition” and “pharmaceutical formulation” (or “formulation”) refers to a preparation which is in such form as to permit the biological activity of an active ingredient contained therein to be effective, and which contains no additional components which are unacceptably toxic to a subject to which the composition would be administered. The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

“filler” refers to an excipient which fills out the size of a tablet or capsule, making it practical to produce and convenient for the consumer to use. Suitable fillers include e.g. pharmaceutically acceptable fillers, such as microcrystalline cellulose (e.g. Avicel®), cellulose powder, Isomalt, lactose, lactose spray-dried, lactose anhydrous, lactose monohydrate, maltodextrin, mannitol, dibasic calcium phosphate, sorbitol, sugars, sucrose, dextrose, sugar alcohols, hydrolyzed starch, corn starch, starch, pregelatinized starch, polysaccharides, dibasic calcium phosphate, calcium sulfate and combination thereof.

As used herein, the term “acidifier” refers to an excipient that is added to a pharmaceutical formulation to lower the pH of the solution or mixture. An acidifier serves to create an acidic environment, which can enhance the stability, solubility, and bioavailability of the active pharmaceutical ingredient (API). Examples of acidifiers may be “organic acidifier” or “inorganic acidifier”. As used herein, the term “organic acidifier” means an acidifier the chemical composition of which contains carbon. As used herein, the term “inorganic acidifier” means an acidifier the composition of which does not contain carbon. In particular, the acidifier, according to the invention, is selected from tartaric acid, malic acid, maleic acid, fumaric acid, citric acid, and betaine hydrochloride. More particularly, the acidifier is tartaric acid, citric acid or malic acid. Even more particularly, the acidifier is tartaric acid. Further more particularly, the acidifier is (D) or (L) tartaric acid or a combination thereof, more particularly (L) Tartaric acid. Further most particularly tartaric acid was found to specifically reduce the pH in the close vicinity of risdiplam, rather than just reduce pH of the solution as a whole generally. By creating a localized acidic environment, the acidifier enhances the solubility of the risdiplam, ensuring its optimal performance. This targeted pH adjustment also at elevated pH conditions. Hence it helps maintain risdiplam efficacy throughout the shelf life of the product. Tartaric acid is incorporated into the formulation to achieve this localized pH reduction, thereby improving the overall therapeutic effectiveness of the medication. Other acidifiers did not show superiority in comparison to tartaric acid

“Pharmaceutically acceptable carrier” refers to an ingredient in a pharmaceutical composition, other than an active ingredient, which is nontoxic to a subject. A pharmaceutically acceptable carrier includes, but is not limited to, a buffer or acidifier, excipient, stabilizer, or preservative.

As used herein, the terms “administration” and “administering” mean the delivery of a bioactive composition or formulation to a subject by an administration route including, but not limited to, oral, intravenous, intra-arterial, intramuscular, intraperitoneal, subcutaneous, intramuscular, topically, or combinations thereof. In some embodiments, the administration to a subject is oral.

“Cₘₐₓ” (expressed in units of pg/mL) refers to the peak concentration that a compound achieves in the plasma of a subject after the compound, or a pharmaceutical composition comprising the compound, has been administrated to the subject. In some embodiments, the compound, or a pharmaceutical composition comprising the compound, is administered orally to a subject to achieve a particular Cₘₐₓ.

“Tₘₐₓ” (expressed in units of hours, or as a median number of hours for Tₘₐₓ in the study population) refers to the time when the peak concentration of a compound in the plasma of a subject is reached after administration of the compound, or a pharmaceutical composition comprising the compound, to the subject. If it occurs at more than one time point Tₘₐₓis defined as the first time point with this value.

“Moisture” refers to the presence of water, including water in trace amounts, steam, humidity, and liquid water.

“OPADRY II white” refers to an iron oxide free film coating comprising polyvinylalcohol, titanium dioxide, Polyethylene Glycol 3350 and Talcum; more particularly for a 2.5 g film coating according to the invention the film coating comprises 1.000 mg polyvinylalcohol, 0.625 mg titanium dioxide, 0.505 mg Polyethylene Glycol 3350 and 0.370 mg Talcum.

“OPADRY II yellow” refers to a film coating comprising polyvinylalcohol, titanium dioxide, Polyethylene Glycol 3350, Talcum and yellow iron oxide; more particularly for a 2.5 g film coating according to the invention the film coating comprises 1.000 mg polyvinylalcohol, 0.608 mg titanium dioxide, 0.505 mg Polyethylene Glycol 3350, 0.370 mg Talcum and 0.017 mg yellow iron oxide.

“x % by weight” or “x±y % by weight” in context with any one of the excipients and/or risdiplam refers to “x±y % by weight” of the tablet Kernel (core tablet) total weight (this total weight is the pharmaceutical composition without the film coating weight) For example, 5 mg of risdiplam in a tablet kernel (core tablet) of 125 mg is 4% by weight of risdiplam of the total kernel weight.

“x % by weight” or “x±y % by weight” in context with any coating agent, colourant, plasticizer and/or anti-tacking agent refers to “x±y % by weight” of a film coating's total weight. For example, 0.61 mg titanium dioxide in the tablet's coating of 2.5 mg is 24.4% by weight of the total weight of the “film coating”, “film coating system”, “film coat” or “coating system”.

“x % by weight” or “x±y % by weight” in context with any one of excipients and/or risdiplam refers to “x±y % by weight” of the coated tablet (Kernel+film coatings). For example, 0.61 mg titanium dioxide in the tablet's coating of 2.5 mg is 24.4% by weight of the total weight of the “film coating”, “film coating system”, “film coat” or “coating system”.

A term like “% w/w” means the weight percentage of the component in respect to the total weight of the composition unless indicated expressly otherwise.

The term “kernel” or “tablet kernel” (also known as “core tablet” or “first layer tablet”) refers to the core composition of the pharmaceutical formulation, excluding any external coatings or layers. The kernel comprises the active pharmaceutical ingredient, in particular risdiplam, along with any excipients that are integral to the formulation's core structure. These excipients may include, but are not limited to ones mentioned herein. The kernel is designed to deliver the API effectively, and its composition is optimized for stability, solubility, and bioavailability.

The term “flowability”, as used herein, is meant to mean and include the ability of a material to move smoothly from one location to another without excessive force, particularly with regard to a powder. The flowability of loose material, in particular of a powder, can be determined by its Flow Factor Coefficient (FFC). The FFC values are known to the skilled person and are also described for example in the article by Dietmar Schulze “Zur Flieβfǎhigkeit von Schüttgütern—Definition und Meβverfahren”, published in the journal “Chemie Ingenieur Technik” by Wiley VCH, 1995, Volume 67, Issue 1, pages 60-68, or in “Powders and Bulk Solids—Behavior, Characterization, Storage and Flow” by Dietmar Schulze, Springer-Verlag Berlin Heidelberg, 2008. The FFC values could be obtained according to http://www.uspbpep.com/usp29/v29240/usp29nf24s0_cl174.htmlit as a USP method as well as a Pheur method. For example, the FFC value can be determined by a uniaxial compression test. In the uniaxial compression test, normally a hollow cylinder, ideally with frictionless walls, is filled with the loose material, in particular with the powder, to be investigated and a stress σ₁—the consolidation stress—is applied in the vertical direction in the first step. Subsequently, the specimen is relieved of the consolidation stress σ₁, and the hollow cylinder is removed. Then, an increasing vertical compressive stress is applied onto the consolidated cylindrical loose material specimen, in particular the consolidated powder specimen, up to the stress σ_{c} at which the cylindrical specimen breaks (or fails). The stress σ_{c} can be called compressive strength or unconfined yield strength. The failure of the consolidated cylindrical specimen upon application of the stress σ_{c} indicates incipient flow of the consolidated loose material, in particular the consolidated powder. The FFC value can then be determined as the ratio FFC=σ_{c}/σ₁.

The term “Direct Compression” refers to a process which involves blending the ingredients directly followed by compaction without the use of a granulation step involving heat and solvent. Preferably, direct compression will be carried out at a temperature of from about ambient to about 45° C., and more preferably from about 20° C. to about 30° C.

As used herein, the term “particle size distribution” or “PSD” means the relative proportions of particles of a compound having a given particle size. While the particle size of a spherical object can be unambiguously and quantitatively defined by its diameter, particles comprising an active pharmaceutical ingredient or an excipient may be non-spherical and irregular in shape. There are several methods by which those of ordinary skill in the art measure and express the size of non-spherical and irregular particles, such as measuring the size of such particles using laser diffractometry and expressing the size of such particles based on replacing a given particle with an imaginary sphere that has one of a number of properties of the particle. Such properties can be selected from, for example, but are not limited to, the diameter of an imaginary sphere having the same volume of the particle being measured (volume-based particle size), the diameter of an imaginary sphere having the same weight as the particle being measured (weight-based particle size), and the diameter of an imaginary sphere having the same surface area as the particle being measured (area-based particle size). Those having ordinary skill in the art are familiar with such methods, and the manner in which the results of such methods are expressed, and such methods can be applied to the embodiments disclosed herein without undue experimentation. The particle size distribution may be represented, for example, graphically as a plot. A common type of plot is a cumulative undersize plot which represents the fraction (e.g. by number, volume or mass) of particles that are smaller than the stated particle size. According to the present invention the PSD is being measure by Laser diffraction.

As used herein, the parameters Dv10, Dv50 and Dv90 represent the particle size at the 10%, 50%, 90% of the cumulative number or volume undersize particle size distribution. Thus, a “Dv10” for a material represents a particle size wherein 10% of the number or volume of the material consists of particles having a particle size equal to the Dv10 value or smaller. A “Dv50” for a material represents a particle size wherein 50% of the number of volume of the material consists of particles having a particle size equal to the Dv50 value or smaller. A “Dv90” for a material represents a particle size wherein 90% of the number or volume of the material consists of particles having a particle size equal to the Dv90 value or smaller. “7-(4,7-diazaspiro[2.5]octan-7-yl)-2-(2,8-dimethylimidazo[1,2-b]pyridazin-6-yl)pyrido[1,2-a]pyrimidin-4-one” refers to a compound of formula (I),

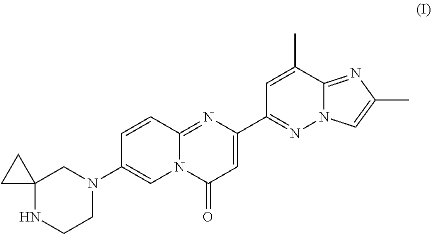 [SMILES: Cc1cn2nc(-c3cc(=O)n4cc(N5CCNC6(CC6)C5)ccc4n3)cc(C)c2n1]

also known as risdiplam, 7-(4,7-diazaspiro[2.5]octan-7-yl)-2-(2,8- dimethylimidazo[1,2-b]pyridazin-6-yl)-4H-pyrido[1,2-a]pyrimidin-4-one, 7-(4,7-diazaspiro[2.5]octan-7-yl)-2-(2,8 dimethylimidazo[1,2-b]pyridazin-6-yl)pyrido-4H-[1,2-a]pyrimidin-4-one, RG7916, RO7034067 or CAS Number 1825352-65-5. Methods of making and using the compound are described in EP3143025 A1. Herein the compound of formula (I)'s name or reference can be interchangeably be used. In a particular embodiment of the invention Risdiplam is in crystalline polymorphic Form A as disclosed in WO2020079203.

WO2017080967 discloses a pharmaceutical composition comprising risdiplam in the form of a powder for oral aqueous solution.

The present invention prevent stability issue and/or minimize the risk of toxicity exposure to the caregiver of the patient. Risdiplam is known to be a highly potent compound. For instance it has been classified by regulatory authorities at EU and national levels to be of 1.0-10 microgram/m3 (IOEL). IEOL are the occupational exposure limit, which are regulatory values which indicate levels of exposure that are considered to be safe (health-based) for a chemical substance in the air of a workplace. The film-coated tablet makes it easier and safer for a caregiver to handle the drug since (1) it is in the form of a tablet, rather than a powder which may be more difficult to handle and come in contact with the caregiver more easily; and (2) a tablet coating prevents dust exposure.

The present invention enable a fast dispersion of the formulation, in particular in 5 ml water, more particular in drinking water with HCl content less than 1 mg/L, even more particular in drinking water with HCl content less than 0.2 mg/L, most particularly wherein the drinking water is mineral water. This ability is critically important for administering the tablet to subjects with limited swallowing ability, and/or those who have physical impairment to swallowing (including impairment as a result of SMA). There are many individuals with SMA who use feeding tubes (e.g., nasogastric tubes) in their care, including adults who may require a 5 mg dose but do not have the ability to swallow a tablet. Thus, dispersion of the formulation is a critical component of certain embodiments of the present disclosure.

To develop a fast dispersion, many different disintegrants were assessed. Furthermore, it was surprisingly found that integration of crospovidone ensures fast disintegration of the tablet whereas other super-disintegrants like sodium croscarmellose lead to incomplete dissolution due to complexation and/or salt formation with Risdiplam.

It was surprisingly found that the present invention overcomes the limited stability of risdiplam when in contact with certain excipients, particularly tartaric acid, in high humidity environment. In particular, it was surprisingly found that risdiplam stability is reduced greatly in presence of or in contact with iron oxide, leading to a change of color of the risdiplam to dark brown. The tablet could change even to black as shown in FIG. 6. This change of color is essentially attributed to chemical reaction of the risdiplam. The present invention address these stability issues of the risdiplam. Notably this is addressed by using at least one film coating without Iron oxide.

It was surprisingly found that risdiplam was incompatible with the presence of Sucralose when exposed to humidity as it promotes decomposition and discoloration as shown in FIG. 3 for instance. Therefore, in a particular embodiment of the invention, the composition as herein described does not comprise sucralose.

All embodiments of present invention can be combined. According to the here within described invention more particular embodiments of the invention are described below:

Embodiment 1. A pharmaceutical composition comprising:
(a) a compound of formula (I)

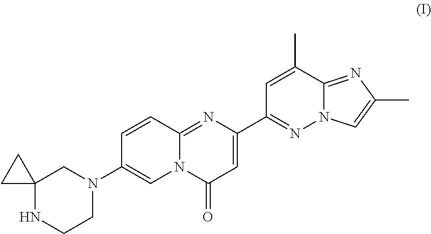 [SMILES: Cc1cn2nc(-c3cc(=O)n4cc(N5CCNC6(CC6)C5)ccc4n3)cc(C)c2n1]

or a pharmaceutically acceptable salt thereof;
(b) crospovidone; and
(c) an acidifier selected from tartaric acid, citric acid or malic acid, more particularly tartaric acid.

Embodiment 2. The pharmaceutical composition according to embodiment 1, comprising
i) a tablet Kernel (also known as core tablet or first layer tablet) comprising:
   (a) a compound of formula (I)

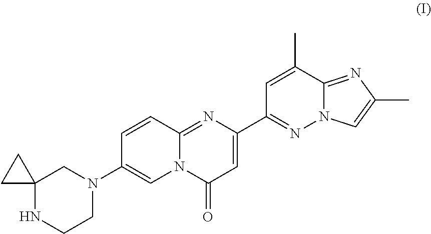 [SMILES: Cc1cn2nc(-c3cc(=O)n4cc(N5CCNC6(CC6)C5)ccc4n3)cc(C)c2n1]

or a pharmaceutically acceptable salt thereof;
(b) crospovidone; and
(c) an acidifier selected from tartaric acid, citric acid or malic acid, more particularly tartaric acid; and
ii) at least one film coating.

Embodiment 3. The pharmaceutical composition according to any one of embodiments 1 to 2, comprising a first film coating.

Embodiment 4. The pharmaceutical composition according to any one of embodiments 1 to 3, comprising a first film coating, wherein the first film coating is coating the tablet kernel (also known as core tablet or first layer tablet (i)) directly.

Embodiment 5. The pharmaceutical composition according to any one of embodiments 2 to 4, wherein at least one film coating, in particular wherein the first film coating is free of iron oxide, in particular where in the film coating, in particular the first film coating, is a polyvinyl alcohol based coat (PVA-based coat), particularly with 5 mg or less PVA-based coat, more particularly with 3.0 mg PVA-based coat, most particularly with 2.5 mg PVA-based coat.

Embodiment 6. The pharmaceutical composition according to any one of embodiments 3 to 4, wherein the first film coating, is free of iron oxide, in particular where in the film coating, in particular the first film coating, is a polyvinyl alcohol based coat (PVA-based coat), particularly with 5 mg or less PVA-based coat, more particularly with 3.0 mg PVA-based coat, most particularly with 2.5 mg PVA-based coat.

Embodiment 7. The pharmaceutical composition according to any one of embodiments 1 to 6, comprising a second film coating, wherein the second film coating is coating the first film coating, in particular where in the second film coating is a polyvinyl alcohol based coat (PVA-based coat), particularly with 5 mg or less PVA-based coat, more particularly with 3.0 mg PVA-based coat, most particularly with 2.5 mg PVA-based coat.

Embodiment 8. The pharmaceutical composition according to any one of embodiments 1 to 7, wherein the composition further comprises a filler selected from microcrystalline cellulose, cellulose powder, Isomalt, lactose (particular example of lactose can be lactose spray-dried, lactose anhydrous or lactose monohydrate), maltodextrin, mannitol, dibasic calcium phosphate, sorbitol, sugars (particular example of sugars cane besucros, dextrose or sugar alcohols), starch (particular example of starch can be hydrolyzed starch, corn starch, or pregelatinized starch), polysaccharides, dibasic calcium phosphate and calcium sulfate, and combinations thereof.

Embodiment 9. The pharmaceutical composition according to any one of embodiments 1 to 8, wherein the composition further comprises a filler selected from lactose, starch (in particular hydrolyzed starch), maltodextrin, Isomalt, microcrystalline cellulose, mannitol, sorbitol, sucrose, dextrose, dibasic calcium phosphate and calcium sulfate, and combinations thereof.

Embodiment 10. The pharmaceutical composition according to any one of embodiments 1 to 9, wherein the composition further comprises as fillers microcrystalline cellulose and mannitol.

Embodiment 11. The pharmaceutical composition according to any one of embodiments 1 to 10, wherein the composition comprises less than 1% by weight, less than 0.1% by weight, less than 0.05% by weight, less than 0.01% by weight, less than 0.005% by weight, or less than 0.001% by weight of disodium ethylenediaminetetraacetate, most particularly no disodium ethylenediaminetetraacetate.

Embodiment 12. The pharmaceutical composition according to any one of embodiments 1 to 11, wherein the composition comprises, less than 0.1% by weight, less than 0.05% by weight, less than 0.01% by weight, less than 0.005% by weight, or less than 0.001% by weight of sucralose, most particularly no sucralose.

Embodiment 13. The pharmaceutical composition according to any one of embodiments 1 to 12, wherein the composition further comprises a lubricant selected from sodium stearyl fumarate and magnesium stearate.

Embodiment 14. The pharmaceutical composition according to any one of embodiments 1 to 13, wherein the composition further comprises a lubricant, wherein the lubricant is sodium stearyl fumarate.

Embodiment 15. The pharmaceutical composition according to any one of embodiments 1 to 14, wherein the composition further comprises a glidant, wherein the glidant is colloidal silicon dioxide.

Embodiment 16. The pharmaceutical composition according to any one of embodiments 1 to 15 comprising:
(a) a compound of formula (I)

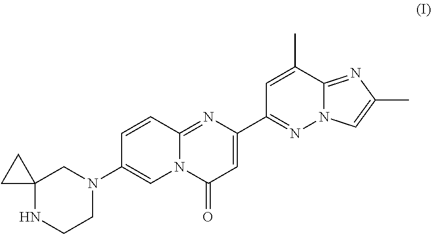 [SMILES: Cc1cn2nc(-c3cc(=O)n4cc(N5CCNC6(CC6)C5)ccc4n3)cc(C)c2n1]

or a pharmaceutically acceptable salt thereof;
(b) crospovidone;
(c) an acidifier selected from tartaric acid, citric acid or malic acid, more particularly tartaric acid; and
(d) at least one filler selected from microcrystalline cellulose, cellulose powder, Isomalt, lactose, lactose spray-dried, lactose anhydrous, lactose monohydrate, maltodextrin, mannitol, dibasic calcium phosphate, sorbitol, sugars, sucrose, dextrose, sugar alcohols, hydrolyzed starch, corn starch, starch, pregelatinized starch, polysaccharides, dibasic calcium phosphate and calcium sulfate and combinations thereof; particularly selected from lactose, starch, hydrolyzed starch, maltodextrin, Isomalt, microcrystalline cellulose, mannitol, sorbitol, sucrose, dextrose, dibasic calcium phosphate and calcium sulfate, and combinations thereof; more particularly a combination of microcrystalline cellulose and mannitol.

Embodiment 17. The pharmaceutical composition according to any one of embodiments 1 to 16 comprising:
(a) a compound of formula (I)

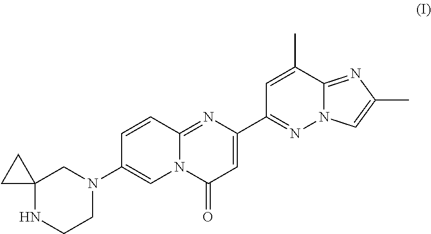 [SMILES: Cc1cn2nc(-c3cc(=O)n4cc(N5CCNC6(CC6)C5)ccc4n3)cc(C)c2n1]

or a pharmaceutically acceptable salt thereof;
(b) crospovidone;
(c) an acidifier selected from tartaric acid; citric acid and malic acid, more particularly tartaric acid;
(d) at least one filler selected from microcrystalline cellulose, cellulose powder, Isomalt, lactose, lactose spray-dried, lactose anhydrous, lactose monohydrate, maltodextrin, mannitol, dibasic calcium phosphate, sorbitol, sugars, sucrose, dextrose, sugar alcohols, hydrolyzed starch, corn starch, starch, pregelatinized starch, polysaccharides, dibasic calcium phosphate and calcium sulfate and combinations thereof, particularly selected from lactose, starch, hydrolyzed starch, maltodextrin, Isomalt, microcrystalline cellulose, mannitol, sorbitol, sucrose, dextrose, dibasic calcium phosphate and calcium sulfate, and combinations thereof, more particularly a combination of microcrystalline cellulose and mannitol; and
(e) a lubricant selected from sodium stearyl fumarate and magnesium stearate, particularly sodium stearyl fumarate.

Embodiment 18. The pharmaceutical composition according to any one of embodiments 1 to 17 comprising:
(a) a compound of formula (I)

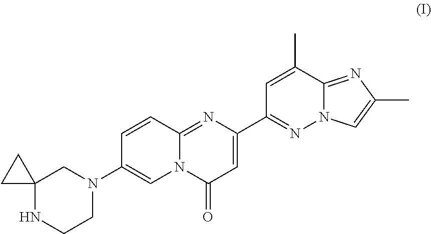 [SMILES: Cc1cn2nc(-c3cc(=O)n4cc(N5CCNC6(CC6)C5)ccc4n3)cc(C)c2n1]

or a pharmaceutically acceptable salt thereof;
(b) crospovidone;
(c) an acidifier selected from tartaric acid, citric acid or malic acid, more particularly tartaric acid;
(d) at least one filler selected from microcrystalline cellulose, cellulose powder, Isomalt, lactose, lactose spray-dried, lactose anhydrous, lactose monohydrate, maltodextrin, mannitol, dibasic calcium phosphate, sorbitol, sugars, sucrose, dextrose, sugar alcohols, hydrolyzed starch, corn starch, starch, pregelatinized starch, polysaccharides, dibasic calcium phosphate and calcium sulfate and combinations thereof, particularly selected from lactose, starch, hydrolyzed starch, maltodextrin, Isomalt, microcrystalline cellulose, mannitol, sorbitol, sucrose, dextrose, dibasic calcium phosphate and calcium sulfate, and combinations thereof, more particularly a combination of microcrystalline cellulose and mannitol;
(e) a lubricant selected from sodium stearyl fumarate; and magnesium stearate, particularly sodium stearyl fumarate; and
(f) a glidant, wherein the glidant is colloidal silicon dioxide.

Embodiment 19. The pharmaceutical composition according to any one of embodiments 1 to 18 comprising:
(a) a compound of formula (I)

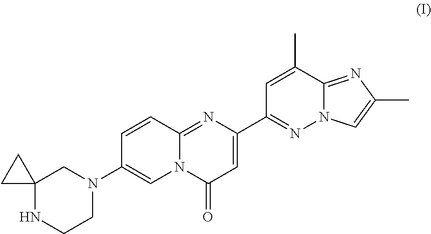 [SMILES: Cc1cn2nc(-c3cc(=O)n4cc(N5CCNC6(CC6)C5)ccc4n3)cc(C)c2n1]

or a pharmaceutically acceptable salt thereof;
(b) crospovidone;
(c) an acidifier selected from tartaric acid, citric acid or malic acid, more particularly tartaric acid;
(d) at least one filler selected from microcrystalline cellulose, cellulose powder, Isomalt, lactose, lactose spray-dried, lactose anhydrous, lactose monohydrate, maltodextrin, mannitol, dibasic calcium phosphate, sorbitol, sugars, sucrose, dextrose, sugar alcohols, hydrolyzed starch, corn starch, starch, pregelatinized starch, polysaccharides, dibasic calcium phosphate and calcium sulfate and combinations thereof, particularly selected from lactose, starch, hydrolyzed starch, maltodextrin, Isomalt, microcrystalline cellulose, mannitol, sorbitol, sucrose, dextrose, dibasic calcium phosphate and calcium sulfate, and combinations thereof, more particularly a combination of microcrystalline cellulose and mannitol;
(e) a lubricant selected from sodium stearyl fumarate and magnesium stearate, particularly sodium stearyl fumarate; and
(f) a glidant wherein the glidant is colloidal silicon dioxide;
   with the proviso that the pharmaceutical composition does not comprise disodium ethylenediaminetetraacetate.

Embodiment 20. The pharmaceutical composition according to any one of embodiments 1 to 19 comprising:
(a) about 5 mg of a compound of formula (I)

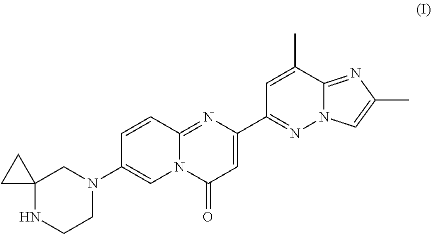 [SMILES: Cc1cn2nc(-c3cc(=O)n4cc(N5CCNC6(CC6)C5)ccc4n3)cc(C)c2n1]

or a pharmaceutically acceptable salt thereof;
(b) crospovidone;
(c) an acidifier selected from tartaric acid, citric acid or malic acid, more particularly tartaric acid;
(d) at least one filler selected from microcrystalline cellulose, cellulose powder, Isomalt, lactose, lactose spray-dried, lactose anhydrous, lactose monohydrate, maltodextrin, mannitol, dibasic calcium phosphate, sorbitol, sugars, sucrose, dextrose, sugar alcohols, hydrolyzed starch, corn starch, starch, pregelatinized starch, polysaccharides, dibasic calcium phosphate and calcium sulfate and combinations thereof, particularly selected from lactose, starch, hydrolyzed starch, maltodextrin, Isomalt, microcrystalline cellulose, mannitol, sorbitol, sucrose, dextrose, dibasic calcium phosphate and calcium sulfate, and combinations thereof, more particularly combination of microcrystalline cellulose and mannitol;
(e) a lubricant selected from sodium stearyl fumarate and magnesium stearate, particularly sodium stearyl fumarate; and
(f) a glidant wherein the glidant is colloidal silicon dioxide;
   with the proviso that the pharmaceutical composition does not comprise disodium ethylenediaminetetraacetate.

Embodiment 21. The pharmaceutical composition according to any one of embodiments 1 to 20, comprising between 0.5% and 30% by weight of risdiplam, particularly between 1% and 15% by weight of risdiplam, more particularly between 2% and 8% by weight of risdiplam, most particularly 4±2% by weight of risdiplam (particularly wherein the weight is the Kernel weight without the coating weight).

Embodiment 22. The pharmaceutical composition according to any one of embodiments 1 to 21, comprising between 2% and 80% by weight of mannitol, particularly between 15% and 45% by weight of mannitol, more particularly between 20% and 40% by weight of mannitol, most particularly 30±5% by weight of mannitol (particularly wherein the weight is the Kernel weight without the coating weight).

Embodiment 23. The pharmaceutical composition according to any one of embodiments 1 to 22 comprising between 0.2% and 5.0% by weight of colloidal silicon dioxide, more particularly between 1.0% and 3.0% by weight of colloidal silicon dioxide, most particularly 2.0±0.5% by weight of colloidal silicon dioxide (particularly wherein the total weight is the Kernel weight, without the coating weight).

Embodiment 24. The pharmaceutical composition according to any one of embodiments 1 to 23 comprising between 0.2% and 5% by weight of sodium stearyl fumarate, more particularly between 2.0% and 4.0% by weight of sodium stearyl fumarate, most particularly 3.0±0.5% by weight of sodium stearyl fumarate (particularly wherein the total weight is the Kernel weight without the coating weight).

Embodiment 25. The pharmaceutical composition according to any one of embodiments 1 to 24 comprising between 5% and 90% by weight of microcrystalline cellulose, particularly between 20% and 60% by weight of microcrystalline cellulose, more particularly 50.0±5% by weight of by weight of microcrystalline cellulose, most particularly 50.0±2% by weight of microcrystalline cellulose (particularly wherein the total weight is the Kernel weight without the coating weight).

Embodiment 26. The pharmaceutical composition according to any one of embodiments 1 to 25 comprising between 0.2% and 12.0% by weight of tartaric acid, more particularly between 1.0% and 4.0% by weight of tartaric acid, most particularly 2.5%±0.5% by weight of tartaric acid (particularly wherein the total weight is the Kernel weight without the coating weight).

Embodiment 27. The pharmaceutical composition according to any one of embodiments 1 to 26 comprising between 0.5% and 12.0% by weight of crospovidone, more particularly between 4.0% and 8.0% by weight of crospovidone, most particularly 6.0%±1.0% by weight of crospovidone (particularly wherein the total weight is the Kernel weight without the coating weight).

Embodiment 28. The pharmaceutical composition according to any one of embodiments 1 to 27, comprising between 0.5% and 30% by weight of risdiplam, particularly between 1% and 15% by weight of risdiplam, more particularly between 2% and 8% by weight of risdiplam, most particularly 4±2% by weight of risdiplam (wherein the total weight is the coated tablet [Kernel weight and the coating weight]).

Embodiment 29. The pharmaceutical composition according to any one of embodiments 1 to 28 comprising between 2% and 80% by weight of mannitol, particularly between 15% and 45% by weight of mannitol, more particularly between 20% and 40% by weight of mannitol, most particularly 30±5% by weight of mannitol (wherein the total weight is the coated tablet [Kernel weight and the coating weight]).

Embodiment 30. The pharmaceutical composition according to any one of embodiments 1 to 29 comprising between 0.2% and 5.0% by weight of colloidal silicon dioxide, more particularly between 1.0% and 3.0% by weight of colloidal silicon dioxide, most particularly 1.90±0.50% by weight of colloidal silicon dioxide (wherein the total weight is the coated tablet [Kernel weight and the coating weight]).

Embodiment 31. The pharmaceutical composition according to any one of embodiments 1 to 30 comprising between 0.2% and 5% by weight of sodium stearyl fumarate, more particularly between 2.0% and 4.0% by weight of sodium stearyl fumarate, most particularly 2.90±0.50% by weight of sodium stearyl fumarate (wherein the total weight is the coated tablet [Kernel weight and the coating weight]).

Embodiment 32. The pharmaceutical composition according to any one of embodiments 1 to 31 comprising between 5% and 90% by weight of microcrystalline cellulose, particularly between 20% and 60% by weight of microcrystalline cellulose, more particularly 50.0±5% by weight of by weight of microcrystalline cellulose, most particularly 48.90±2.0% by weight of microcrystalline cellulose (wherein the total weight is the coated tablet [Kernel weight and the coating weight]).

Embodiment 33. The pharmaceutical composition according to any one of embodiments 1 to 32 comprising between 0.2% and 12.0% by weight of tartaric acid, more particularly between 1.0% and 4.0% by weight of tartaric acid, most particularly 2.30%±0.5% by weight of tartaric acid (wherein the total weight is the coated tablet [Kernel weight and the coating weight]).

Embodiment 34. The pharmaceutical composition according to any one of embodiments 1 to 33 comprising between 0.5% and 12.0% by weight of crospovidone, more particularly between 4.0% and 8.0% by weight of crospovidone, most particularly 5.80%±1.0% by weight of crospovidone (wherein the total weight is the coated tablet [Kernel weight and the coating weight]).

Embodiment 35. The pharmaceutical composition according to any one of embodiments 1 to 34 comprising:
(a) between 0.5% and 30% by weight of, particularly between 1% and 15% by weight of, more particularly between 2% and 8% by weight of, most particularly 4±2% by weight of a compound of formula (I)

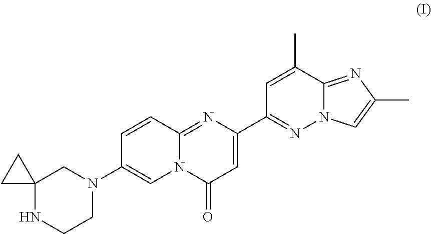 [SMILES: Cc1cn2nc(-c3cc(=O)n4cc(N5CCNC6(CC6)C5)ccc4n3)cc(C)c2n1]

or a pharmaceutically acceptable salt thereof;
(b) between 0.5% and 12.0% by weight of crospovidone, more particularly between 4.0% and 8.0% by weight of crospovidone, most particularly 6.0%±1.0% by weight of crospovidone;
(c) between 0.2% and 12.0% by weight of, more particularly between 1.0% and 4.0% by weight of, most particularly 2.5%±0.5% by weight of an acidifier selected from tartaric acid, citric acid or malic acid, more particularly tartaric acid;
(d) between 30% and 90% by weight, particularly between 40% and 90% by weight of, between 70% and 90% by weight of, most particularly 80±5% by weight of filler, wherein the filler comprises at least one filler selected from microcrystalline cellulose, cellulose powder, Isomalt, lactose, lactose spray-dried, lactose anhydrous, lactose monohydrate, maltodextrin, mannitol, dibasic calcium phosphate, sorbitol, sugars, sucrose, dextrose, sugar alcohols, hydrolyzed starch, corn starch, starch, pregelatinized starch, polysaccharides, dibasic calcium phosphate and calcium sulfate and combinations thereof, particularly selected from lactose, starch, hydrolyzed starch, maltodextrin, Isomalt, microcrystalline cellulose, mannitol, sorbitol, sucrose, dextrose, dibasic calcium phosphate and calcium sulfate, and combinations thereof, more particularly combination of microcrystalline cellulose and mannitol;
(e) between 0.2% and 5% by weight of, more particularly between 2.0% and 4.0% by weight of, most particularly 3.0±0.5% of a lubricant selected from sodium stearyl fumarate and magnesium stearate, particularly sodium stearyl fumarate; and
(f) between 0.2% and 5.0% by weight of, more particularly between 1.0% and 3.0% by weight of, most particularly 2.0±0.5% by weight of a glidant, wherein the glidant is colloidal silicon dioxide;
   with the proviso that the pharmaceutical composition does not comprise disodium ethylenediaminetetraacetate;
   wherein the total amount of ingredients does not exceed 100% weight (particularly wherein the weight is the Kernel weight without the coating weight).

Embodiment 36. The pharmaceutical composition according to any one of embodiments 1 to 35 comprising:
(a) between 0.5% and 30% by weight of, particularly between 1% and 15% by weight of, more particularly between 2% and 8% by weight of, most particularly 4±2% by weight of a compound of formula (I)

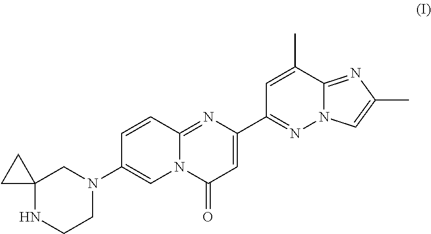 [SMILES: Cc1cn2nc(-c3cc(=O)n4cc(N5CCNC6(CC6)C5)ccc4n3)cc(C)c2n1]

or a pharmaceutically acceptable salt;
(b) between 0.5% and 12.0% by weight of crospovidone, more particularly between 4.0% and 8.0% by weight of crospovidone, most particularly 6.0%±1.0% by weight of crospovidone;
(c) between 0.2% and 12.0% by weight of, more particularly between 1.0% and 4.0% by weight of, most particularly 2.5%±0.5% by weight of an acidifier selected from tartaric acid, citric acid or malic acid, more particularly tartaric acid;
(d) between 5% and 90% by weight of, particularly between 20% and 60% by weight of, more particularly 50.0±5% by weight of by weight of, most particularly 50.0±2% by weight of microcrystalline cellulose;
(e) between 2% and 80% by weight of, particularly between 15% and 45% by weight of, more particularly between 20% and 40% by weight of, most particularly 30±5% by weight of mannitol;
(f) between 0.2% and 5% by weight of, more particularly between 2.0% and 4.0% by weight of, most particularly 3.0±0.5% of sodium stearyl fumarate; and
(g) between 0.2% and 5.0% by weight of, more particularly between 1.0% and 3.0% by weight of, most particularly 2.0±0.5% by weight of colloidal silicon dioxide; with the proviso that the pharmaceutical composition does not comprise disodium ethylenediaminetetraacetate;
   wherein the total amount of ingredients does not exceed 100% weight (particularly wherein the weight is the Kernel weight without the coating weight).

Embodiment 37. The pharmaceutical composition according to any one of embodiments 1 to 36 comprising:
i) a tablet Kernel (also known as core tablet or first layer tablet) comprising:
   (a) between 0.5% and 30% by weight of, particularly between 1% and 15% by weight of, more particularly between 2% and 8% by weight of, most particularly 4±2% by weight of a compound of formula (I)

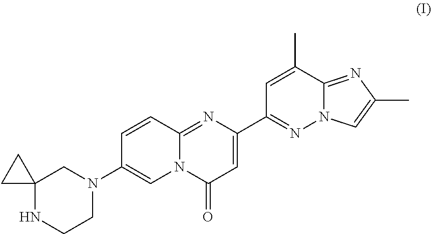 [SMILES: Cc1cn2nc(-c3cc(=O)n4cc(N5CCNC6(CC6)C5)ccc4n3)cc(C)c2n1]

or a pharmaceutically acceptable salt;
(b) between 0.5% and 12.0% by weight of crospovidone, more particularly between 4.0% and 8.0% by weight of crospovidone, most particularly 5.8%±1.0% by weight of crospovidone;
(c) between 0.2% and 12.0% by weight of, more particularly between 1.0% and 4.0% by weight of, most particularly 2.3%±0.5% by weight of an acidifier selected from tartaric acid, citric acid or malic acid, more particularly tartaric acid;
(d) between 5% and 90% by weight of, particularly between 20% and 60% by weight of, more particularly 50.0±5% by weight of by weight of, most particularly 48.9±2% by weight of microcrystalline cellulose;
(e) between 2% and 80% by weight of, particularly between 15% and 45% by weight of, more particularly between 20% and 40% by weight of, most particularly 30±5% by weight of mannitol;
(f) between 0.2% and 5% by weight of, more particularly between 2.0% and 4.0% by weight of, most particularly 2.9±0.5% of sodium stearyl fumarate; and
(g) between 0.2% and 5.0% by weight of, more particularly between 1.0% and 3.0% by weight of, most particularly 1.9±0.5% by weight of colloidal silicon dioxide; and
ii) at least one film coating;
   with the proviso that the pharmaceutical composition does not comprise disodium ethylenediaminetetraacetate;
   wherein the total amount of ingredients does not exceed 100% weight.

Embodiment 38. The pharmaceutical composition according to any one of embodiments 1 to 37 comprising:
i) a tablet Kernel (also known as core tablet or first layer tablet) comprising:
   (a) between 0.5% and 30% by weight of, particularly between 1% and 15% by weight of, more particularly between 2% and 8% by weight of, most particularly 4±2% by weight of a compound of formula (I)

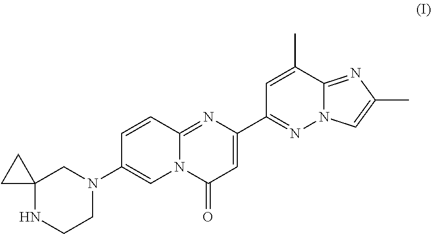 [SMILES: Cc1cn2nc(-c3cc(=O)n4cc(N5CCNC6(CC6)C5)ccc4n3)cc(C)c2n1]

or a pharmaceutically acceptable salt;
(b) between 0.5% and 12.0% by weight of crospovidone, more particularly between 4.0% and 8.0% by weight of crospovidone, most particularly 5.8%±1.0% by weight of crospovidone;
(c) between 0.2% and 12.0% by weight of, more particularly between 1.0% and 4.0% by weight of, most particularly 2.3%±0.5% by weight of an acidifier selected from tartaric acid, citric acid or malic acid, more particularly tartaric acid;
(d) between 5% and 90% by weight of, particularly between 20% and 60% by weight of, more particularly 50.0±5% by weight of by weight of, most particularly 48.9±2.0% by weight of microcrystalline cellulose;
(e) between 2% and 80% by weight of, particularly between 15% and 45% by weight of, more particularly between 20% and 40% by weight of, most particularly 30±5% by weight of mannitol;
(f) between 0.2% and 5% by weight of, more particularly between 2.0% and 4.0% by weight of, most particularly 2.9±0.50% of sodium stearyl fumarate; and
(g) between 0.2% and 5.0% by weight of, more particularly between 1.0% and 3.0% by weight of, most particularly 1.9±0.50% by weight of colloidal silicon dioxide; and
(ii) first film coating, wherein the first film coating is coating the core tablet (i) directly and second film coating is coating the first film coating;
with the proviso that the pharmaceutical composition does not comprise disodium ethylenediaminetetraacetate
wherein the total amount of ingredients does not exceed 100% weight.

Embodiment 39. The pharmaceutical composition according to any one of embodiments 1 to 38, wherein sodium stearyl fumarate has a saponification value of 142.2 to 146.0, particularly has a Dv50 of 13.6 μm, more particularly sodium stearyl fumarate is Pruv®.

Embodiment 40. The pharmaceutical composition according to any one of embodiments 1 to 39, wherein microcrystalline cellulose is CAS 9004-34-6, particularly has a Dv50 135 μm, more particularly is PH102.

Embodiment 41. The pharmaceutical composition according to any one of embodiments 1 to 40, comprises only one active pharmaceutical ingredient (API), more particularly wherein the only API is the compound of formula (I)

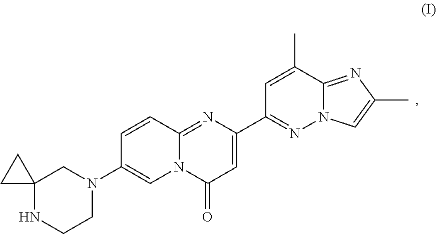 [SMILES: Cc1cn2nc(-c3cc(=O)n4cc(N5CCNC6(CC6)C5)ccc4n3)cc(C)c2n1]

also known as risdiplam.

Embodiment 42. A tablet comprising the pharmaceutical composition in according to any one of embodiments 1 to 41.

Embodiment 43. The tablet according to embodiment 42, wherein the tablet is dispersible in water within 5 minutes.

Embodiment 44. The tablet according to any one of embodiments 42 to 43, wherein the tablet is dispersible in water with HCl content less than 1 mg/L, even more particular in drinking water with HCl content less than 0.2 mg/L, most particularly wherein the drinking water is mineral water.

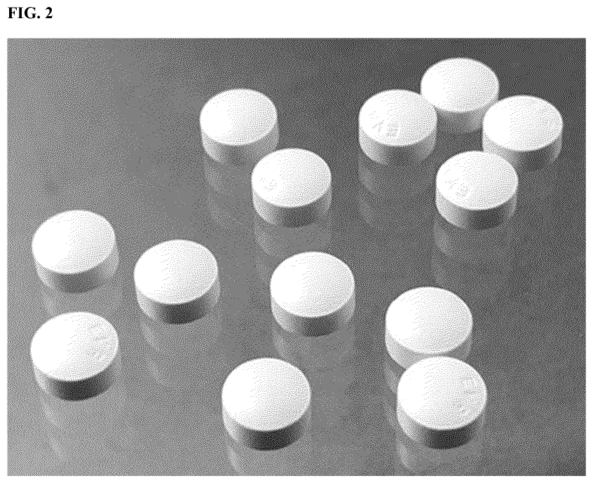

Embodiment 45. The tablet according to any one of embodiments 42 to 44 having a shape as drawn in FIG. 2, more particularly wherein the tablet has a diameter of 6.50±0.50 mm.

Embodiment 46. A kit comprising the pharmaceutical composition according to any one of embodiments 1 to 41, in the form of a tablet comprising a therapeutically effective amount of risdiplam, prescribing information also known as “leaflet”, a blister package or bottle (HDPE or glass), particularly a moisture protective primary packaging, more particularly Alu/Alu blister or a plastic bottle with desiccant and a container, in particular wherein the prescribing information particularly includes the advice to a patient regarding the administration of the risdiplam with food.

Embodiment 47. A process to produce the pharmaceutical composition according to any one of embodiments 1 to 41, comprising the following steps:
i) blend the compound of formula (I)

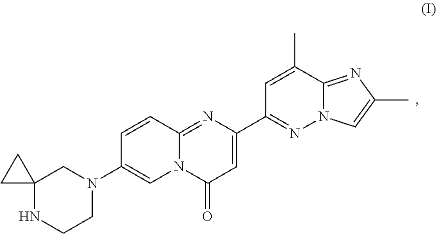 [SMILES: Cc1cn2nc(-c3cc(=O)n4cc(N5CCNC6(CC6)C5)ccc4n3)cc(C)c2n1]

also known as risdiplam, with tartaric acid (particularly as fine powder), mannitol, microcrystalline cellulose, colloidal silicon dioxide, crospovidone and strawberry flavor in container 1;
ii) sieve of container 1,
iii) sieve sodium stearyl fumarate, in particular to 0.5 mm, in container 2, iv) blend the mixture of container 1 and container 2;
v) compress the blend of iv) into tablet kernels;
vi) spray a first film-coating system onto the tablet kernels to form a first film coating, wherein the first film-coating system comprises a suspension of OPADRY II white in purified water, wherein the first film-coating system is substantially free of iron oxide; and
vii) spray a second film-coating system onto the first film coating to form a second film coat, wherein the second film-coating system comprises a suspension of OPADRY II yellow in purified water.

Embodiment 48. A process to produce the pharmaceutical composition according to any one of embodiments 1 to 41, comprising the following steps:
viii) blend the compound of formula (I)

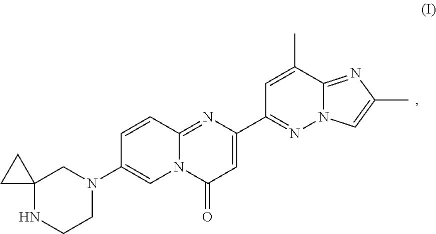 [SMILES: Cc1cn2nc(-c3cc(=O)n4cc(N5CCNC6(CC6)C5)ccc4n3)cc(C)c2n1]

also known as risdiplam with tartaric acid powder, mannitol, Microcrystalline cellulose, colloidal silicon dioxide, crospovidone and strawberry flavor in container 1;
ix) sieve of container 1,
x) sieve sodium stearyl fumarate, in particular to 0.5 mm, in container 2,
xi) blend the mixture of container 1 and container 2;
xii) compress the blend of iv) into tablet kernels, and
xiii) suspend the first film-coating system, the film-coating system, in particular wherein the film-coating system is free of iron oxide, more particularly where film-coating system is OPADRY II white (i.e. 85F18422), into purified water, and
xiv) spray the film coating system vi) onto the tablet kernels.
xv) suspend the film-coating system, in particular wherein the film-coating system does not comprise iron oxide, more particularly where film-coating system is OPADRY II yellow (i.e. 85F220022), into purified water, and
xvi) spray the film coating system viii) onto the coated tablet kernels.

Embodiment 49. A pharmaceutical composition obtained by the process according to embodiment 47 or 48.

Embodiment 50. A pharmaceutical composition according to any one of embodiments 1 to 41, comprising risdiplam that is administered to an individual at any suitable dosage (e.g. to achieve a therapeutically effective amount), particularly a suitable dose of a therapeutically effective amount of about 5 mg per day.

Embodiment 51. The use of a pharmaceutical composition according to any one of embodiments 1 to 41, for the treatment, prevention, delaying progression, and/or amelioration of diseases caused by an inactivating mutation or deletion in the SMN1 gene and/or associated with loss or defect of SMN1 gene function, particularly for the treatment, prevention, delaying progression and/or amelioration of spinal muscular atrophy (SMA), in particular in human subject in need thereof.

Embodiment 52. The use of a pharmaceutical composition according to any one of embodiments 1 to 41, for the preparation of medicaments for the treatment, prevention, delaying progression, and/or amelioration of diseases caused by an inactivating mutation or deletion in the SMN1 gene and/or associated with loss or defect of SMN1 gene function, particularly for the treatment, prevention, delaying progression and/or amelioration of spinal muscular atrophy (SMA) in particular in human subject in need thereof.

Embodiment 53. A pharmaceutical composition according to any one of embodiments 1 to 41 for the treatment, prevention, delaying progression, and/or amelioration of diseases caused by an inactivating mutation or deletion in the SMN1 gene and/or associated with loss or defect of SMN1 gene function, particularly for the treatment, prevention, delaying progression and/or amelioration of spinal muscular atrophy (SMA), in particular in human subject in need thereof.

Embodiment 54. A pharmaceutical composition according to embodiment 53 for the treatment, wherein the patients have a body weight of more than or equal to 20 kg.

Embodiment 55. A pharmaceutical composition according to embodiment 53 for the treatment, wherein the human subject has a body weight of more than or equal to 20 kg.

In a more particular embodiment, the invention relates to fast disintegrating film-coated tablet which promotes complete dissolution of Risdiplam once the tablet is dispersed in a small amount of water (for example, 5 ml) based on the presence of the specific amount of tartaric acid as an acidifier.

In some embodiments, the tablet comprises a filler. In a particular embodiment of the invention, the fillers are mannitol, particularly D-mannitol suitable for direct compression such as Parteck® M100 in combination with Microcrystalline Cellulose, particularly type 102, to promote good flowability and homogenous distribution of the API for a direct compression process.

In a particular embodiment of the invention, crospovidone comprises low amount of nitrite (less than 100 ppm by weight of nitrite, particularly less than 50 ppm by weight of nitrite, more particularly less than 35 ppm by weight of nitrite, most particularly less than 0.1 ppm of nitrite)

In some embodiments, provided herein is a pharmaceutical formulation comprising a tablet core, wherein the tablet core comprises Risdiplam, an acidifier, and crospovidone. In some embodiments of the tablet provided herein, Risdiplam is present at between 2 and 6% w/w, between 3 and 5% w/w, between 3 and 4% w/w, between 3.5 and 4% w/w, or about 4% w/w. The amount of Ridiplam present may be, for example, relative to the tablet core, or relative to the entire tablet formulation (for example, if the formulation comprises additional components). Thus, in some embodiments Risdiplam is present at between 2 and 6% w/w, between 3 and 5% w/w, between 3 and 4% w/w, between 3.5 and 4% w/w, or about 4% w/w, relative to the tablet core. In some embodiments, Risdiplam is present at between 2 and 6% w/w, between 3 and 5% w/w, between 3 and 4% w/w, between 3.5 and 4% w/w, or about 3.85% w/w, relative to the entire tablet formulation. In some embodiments, the acidifier is an organic acid. In some embodiments, the acidifier is selected from tartaric acid, maleic acid, and citric acid. In some embodiments, the acidifier is tartaric acid. The acidifier may be present, for example, at a ratio relative to Risdiplam of about 2:1 to 1:4, or about 1:1 to 1:3, or about 1:1 to 1:2, or about 1:1.5 to about 1:2, or about 3:5. In some embodiments, the acidifier is present at a weight of between 0.5 to 8% w/w, or between 1 to 7% w/w, or between 1 to 5% w/w, or between 1 to 4% w/w, or between 2 to 3% w/w, or about 2.4% w/w or about 2.3% w/w. The amount of acidifier present may be, for example, relative to the tablet core, or relative to the entire tablet formulation (for example, if the formulation comprises additional components). Thus, in some embodiments the acidifier is present at a weight of between 0.5 to 5% w/w, or between 1 to 4% w/w, or between 2 to 3% w/w, or about 2.4% w/w, relative to the tablet core. In other embodiments, the acidifier is present at a weight of between 0.5 to 5% w/w, or between 1 to 4% w/w, or between 2 to 3% w/w, or about 2.3% w/w relative to the entire tablet formulation. The tablet further comprises crospovidone. In some embodiments, the crospovidone is present at a ratio relative to Risdiplam of about 4:1 to 1:6, or about 3:1 to 1:3, or about 3:1 to 1:1, or about 3:2. In some embodiments, the crospovidone is present at a weight of between 1 to 10% w/w, or between 2 to 8% w/w, or between 4 to 8% w/w, or about 6% w/w or about 5.7% w/w. The amount of crospovidone present may be, for example, relative to the tablet core, or relative to the entire tablet formulation (for example, if the formulation comprises additional components). Thus in some embodiments, the crospovidone is present at a weight of between 1 to 10% w/w, or between 2 to 8% w/w, or between 4 to 8% w/w, or about 6% w/w relative to the tablet core. In other embodiments, the crospovidone is present at a weight of between 1 to 10% w/w, or between 2 to 8% w/w, or between 4 to 8% w/w, or about 5.7% w/w relative to the entire tablet formulation.

In some embodiments, the tablet core further comprises a filler. In embodiments comprising a filler, the filler may be present, for example, in a range from 50 to 90% w/w, or between 60 to 90% w/w, or between 70 to 90% w/w, or between 75 to 85% w/w, relative to the tablet core or to the entire tablet formulation. In some embodiments, the filler is selected from microcrystalline cellulose, cellulose powder, isomalt, lactose (such as lactose spray-dried, lactose anhydrous, or lactose monohydrate), sugars (such as sucrose or dextrose), sugar alcohol (such as mannitol or sorbitol), starch (such as hydrolyzed starch, corn starch, or pregelatinized starch), polysaccharides (such as maltodextrin), dibasic calcium phosphate, and calcium sulfate, and any combinations thereof. In some embodiments, the filler is cellulose and sugar alcohol. In some embodiments, the filler is microcrystalline cellulose and mannitol. In certain particular embodiments, the filler is D-mannitol suitable for direct compression such as Parteck® M100; and microcrystalline cellulose, particularly type 102.

In some embodiments, the tablet core further comprises a lubricant. The lubricant may be present, for example, in a range from 1 to 5% w/w, or between 2 to 4% w/w, or between 2.5 to 3.5% w/w, or about 3% w/w, or about 2.88% w/w, relative to the tablet core or the entire tablet formulation. In some embodiments, the lubricant is selected from sodium stearyl fumarate and magnesium stearate. In some embodiments, the lubricant is sodium stearyl fumarate.

In some embodiments, the tablet core further comprises a glidant. The glidant may be present, for example in a range from 0.2 to 5% w/w, or between 0.5 to 4% w/w, or between 1 to 3% w/w, or between 1.5 to 2.5% w/w, or about 2% w/w, or about 1.92% w/w, relative to the tablet core or the entire table formulation. In some embodiments, the glidant is silicon dioxide. In some embodiments, the glidant is colloidal silicon dioxide.

In some embodiments, the tablet core further comprises a flavoring agent. Such flavoring agent may be present in an amount from 0.02 to 0.2% w/w, or 0.05 to 0.15% w/w, such as about 0.1% w/w.

In some embodiments, the pharmaceutical composition is essentially free of disodium ethylenediaminetetraacetate (which may also be known as disodium EDTA, or edetate disodium). For example, in some embodiments the pharmaceutical composition comprises less than 1% w/w, less than 0.1% w/w, less than 0.05% w/w, less than 0.01% w/w, less than 0.005% w/w, or less than 0.001% w/w disodium ethylenediaminetetraacetate, relative to the entire tablet formulation. In some embodiments, the level of disodium ethylenediaminetetraacetate in the pharmaceutical composition is below detection levels.

In some embodiments, the pharmaceutical composition is essentially free of sucralose. For example, in some embodiments the pharmaceutical composition comprises less than less than 0.1% w/w, less than 0.05% w/w, less than 0.01% w/w, less than 0.005% w/w, or less than 0.001% w/w sucralose, relative to the entire tablet formulation. In some embodiments, the level of sucralose in the pharmaceutical composition is below detection levels.

In some embodiments, the pharmaceutical composition further comprises one or more layers coating the tablet core. In some embodiments, the pharmaceutical composition comprises at least one coating layer, which may otherwise be called a first film coating. In some embodiments, the first film coating directly contacts the tablet core. For example, the first film coating may be sprayed directly onto the tablet core. In some embodiments, the first film coating is essentially free of iron oxides. For example, the first film coating may comprise less than 0.1% w/w, less than 0.05% w/w, less than 0.01% w/w, less than 0.005% w/w, or less than 0.001% w/w iron oxide. In some embodiments, the level of iron oxide in the first film coating is below detection levels. In some embodiments, the first coating layer comprises polyvinyl alcohol (PVA), for example comprises between 30 and 50% w/w, or about 40% PVA. In some embodiments, the first coating layer further comprises polyethylene glycol (PEG), for example comprises between 10% and 30% PEG, or about 20% PEG. In some embodiments, the first coating layer further comprises titanium dioxide, or talcum, or a combination thereof. In some embodiments of a pharmaceutical composition comprising a first film coating as described herein, the tablet kernel is coated with an amount of film coating less than 5% w/w, less than 4% w/w, less than 3% w/w, about 2% w/w, between 0.5 and 5% w/w, between 0.5 and 4% w/w, or between 1 and 3% w/w of the uncoated tablet kernel. Thus for example, a 125 mg tablet kernel coated with 2.5 mg first film coating comprises a coating of 2% w/w of the uncoated tablet kernel. In some embodiments, the first film coating comprises less than 5% w/w, less than 4% w/w, less than 3% w/w, about 2% w/w, between 0.5 and 5% w/w, between 0.5 and 4% w/w, or 1 and 3% w/w, or between 1.5 and 2.5% w/w of the total weight of the coated tablet.

In some embodiments, the pharmaceutical composition comprises a plurality of coatings. For example, in some embodiments, the pharmaceutical composition comprises a first film coating and a second film coating, wherein the first film coating is in direct contact with the tablet kernel and the second film coating coats the first film coating. In some embodiments, the second film coating is in direct contact with the first film coating. In other embodiments, there are one or more intermediate coatings between the first and second film coatings. In some embodiments, the second film coating comprises one or more iron oxides, for example yellow iron oxide. In some embodiments, the second film coating comprises between 0.01 and 1% w/w iron oxide, such as yellow iron oxide. In some embodiments, the second film coating comprises polyvinyl alcohol (PVA), for example comprises between 30 and 50% w/w, or about 40% PVA. In some embodiments, the second film coating further comprises polyethylene glycol (PEG), for example comprises between 10% and 30% PEG, or about 20% PEG. In some embodiments, the second film coating further comprises titanium dioxide, or talcum, or a combination thereof. In some embodiments of a pharmaceutical composition comprising a second film coating as described herein, the tablet kernel is coated with an amount of second coating (for example, coating a first film coating that is also present) less than 5% w/w, less than 4% w/w, less than 3% w/w, about 2% w/w, between 0.5 and 5% w/w, between 0.5 and 4% w/w, or between 1 and 3% w/w of the uncoated tablet kernel. Thus for example, a 125 mg tablet kernel coated with 2.5 mg second film coating comprises a coating of 2% w/w of the uncoated tablet kernel. In some embodiments, the second film coating comprises less than 5% w/w, less than 4% w/w, less than 3% w/w, about 2% w/w, between 0.5 and 5% w/w, between 0.5 and 4% w/w, or 1 and 3% w/w, or between 1.5 and 2.5% w/w of the total weight of the coated tablet. In some embodiments, the amount of first and second film coatings is about the same, wherein the first film coating is directly contacting the tablet kernel and the second film coating coats the first film coating, for example directly contacts the first film coating.

In some embodiments, provided herein is a pharmaceutical formulation comprising a tablet kernel, wherein the tablet kernel comprises Risdiplam, an acidifier, and crospovidone, wherein: Risdiplam is present at between 2 and 6% w/w; the acidifier is present at between 0.5 to 8% w/w; and the crospovidone is present at between 1 to 10% w/w, relative to the tablet kernel. In some embodiments, the acidifier is present at between 1 to 7% w/w relative to the tablet kernel. In some embodiments, the acidifier is a carboxylic acid, such as one selected from tartaric acid, maleic acid, and citric acid. In some embodiments, the acidifier is tartaric acid. In some embodiments, the tablet comprises about 5 mg of Risdiplam. In some embodiments, the tablet kernel further comprises between 70 to 90% w/w filler; between 1 to 5% w/w lubricant; and between 0.5 to 4% w/w glidant, relative to the tablet kernel. In some embodiments, the filler is cellulose and sugar alcohol, for example microcrystalline cellulose and mannitol, such as D-mannitol and type 102 microcrystalline cellulose. In some embodiments, the lubricant is selected from sodium stearyl fumarate and magnesium stearate, for example is sodium stearyl fumarate. In some embodiments, the glidant is silicon dioxide, such as colloidal silicon dioxide. In certain embodiments, the pharmaceutical formulation further comprises a first film coating, wherein the first film coating directly contacts the tablet kernel and is essentially free of iron oxide. In some embodiments, the first film coating comprises PVA, for example comprises between 30 and 50% w/w. In some embodiments the first film coating further comprises PEG, such as between 10% and 30% PEG. In some embodiments the first film coating further comprises titanium dioxide, or talcum, or a combination thereof. In some embodiments the first film coating comprises less than 5% w/w, such as between 1 and 3% w/w, of the total weight of the coated tablet. In some embodiments the pharmaceutical composition further comprises a second film coating comprising iron oxide, wherein the second film coating coats the first film coating, for example directly contacts the first film coating. In some embodiments the second film coating comprises between 0.01 and 1% w/w iron oxide, such as yellow iron oxide. In some embodiments, the second film coating comprises PVA, for example comprises between 30 and 50% w/w. In some embodiments the second film coating further comprises PEG, such as between 10% and 30% PEG. In some embodiments the second film coating further comprises titanium dioxide, or talcum, or a combination thereof. In some embodiments the second film coating comprises less than 5% w/w, such as between 1 and 3% w/w, of the total weight of the coated tablet (including first and second coatings).

The pharmaceutical composition of the present invention which comprises risdiplam possess valuable pharmacological properties and has been found to enhance inclusion of exon 7 of SMN1 and/or SMN2 into mRNA transcribed from the SMN1 and/or SMN2 gene, thereby increasing expression of SMN protein in a human subject in need thereof.

The pharmaceutical composition of the present invention can be used, either alone or in combination with other drugs, for the treatment, prevention, delaying progression and/or amelioration of diseases caused by an inactivating mutation or deletion in the SMN1 gene and/or associated with loss or defect of SMN1 gene function. These diseases include, but are not limited to spinal muscular atrophy (SMA).

A particular embodiment of the present invention relates to a pharmaceutical composition as defined herein for the treatment, prevention, delaying progression and/or amelioration of diseases caused by an inactivating mutation or deletion in the SMN1 gene and/or associated with loss or defect of SMN1 gene function, particularly for the treatment, prevention, delaying progression and/or amelioration of SMA.

A particular embodiment of the present invention relates to a pharmaceutical composition relates to a pharmaceutical composition as defined herein for use as therapeutically active substances, especially for use as therapeutically active substances for the treatment, prevention, delaying progression and/or amelioration of diseases caused by an inactivating mutation or deletion in the SMN1 gene and/or associated with loss or defect of SMN1 gene function, particularly for the treatment, prevention, delaying progression and/or amelioration of spinal muscular atrophy (SMA).

A particular embodiment of the present invention relates to a pharmaceutical composition relates to a pharmaceutical composition as defined herein for the use in the treatment, prevention, delaying progression and/or amelioration of diseases caused by an inactivating mutation or deletion in the SMN1 gene and/or associated with loss or defect of SMN1 gene function, particularly for use in the treatment, prevention, delaying progression and/or amelioration of spinal muscular atrophy (SMA).

A particular embodiment of the present invention relates to a method for the treatment, prevention, delaying progression and/or amelioration of diseases caused by an inactivating mutation or deletion in the SMN1 gene and/or associated with loss or defect of SMN1 gene function, particularly for the treatment, prevention, delaying progression and/or amelioration of spinal muscular atrophy (SMA), which method comprises administering a pharmaceutical composition relates to a pharmaceutical composition as defined herein to a subject.

A particular embodiment of the present invention relates to the use of a pharmaceutical composition relates to a pharmaceutical composition as defined herein for the treatment, prevention, delaying progression and/or amelioration of diseases caused by an inactivating mutation or deletion in the SMN1 gene and/or associated with loss or defect of SMN1 gene, particularly for the treatment, prevention, delaying progression and/or amelioration of spinal muscular atrophy (SMA).

A particular embodiment of the present invention relates to the use of a pharmaceutical composition relates to a pharmaceutical composition as defined herein, for the preparation of medicaments for the treatment, prevention, delaying progression and/or amelioration of diseases caused by an inactivating mutation or deletion in the SMN1 gene and/or associated with loss or defect of SMN1 gene function, particularly for the treatment, prevention, delaying progression and/or amelioration of spinal muscular atrophy (SMA).

In general, the nomenclature used in this Application is based on AUTONOM™ 2000, a Beilstein Institute computerized system for the generation of IUPAC systematic nomenclature. Chemical structures shown herein were prepared using MDL ISIS™ version 2.5 SP2. Any open valency appearing on a carbon, oxygen or nitrogen atom in the structures herein indicates the presence of a hydrogen atom.

### Examples

The invention will be more fully understood by reference to the following examples. They should however not be construed as limiting the scope of the invention.

### Example 1: Impact of the Acidifier Concentration on Risdiplam Dissolution

The below mentioned formulations were prepared in accordance to the mentioned process by varying the amount of Tartaric Acid.

The tablets were produced on 125 g lab scale by a subsequent manual sieving, Turbula blending of all excipients except lubricant, manual sieving and addition of the lubricant and final Turbula blending process followed by tablet compression using a Styl'One single punch press and a target main compression force between 4-4.65 kN.

TABLE 1
Composition of the Tablets used in example 1

| | Formulation 6 | | Formulation 5 | | Formulation 4 | | Formulation 3 | | Formulation 2 | | Formulation 1 | |
|---|---|---|---|---|---|---|---|---|---|---|---|---|
| | [mg] | [%] | [mg] | [%] | [mg] | [%] | [mg] | [%] | [mg] | [%] | [mg] | [%] |
| Risdiplam | 5.00 | 4.00 | 5.00 | 4.00 | 5.00 | 4.00 | 5.00 | 4.00 | 5.00 | 4.00 | 5.00 | 4.00 |
| Tartaric Acid | 7.53 | 6.02 | 6.00 | 4.80 | 4.00 | 3.20 | 3.00 | 2.40 | 2.00 | 1.60 | — | — |
| Microcrystalline | 60.72 | 48.57 | 61.66 | 49.32 | 62.89 | 50.31 | 63.51 | 50.80 | 64.12 | 51.29 | 61.66 | 49.32 |
| Cellulose | | | | | | | | | | | | |
| Mannitol | 37.88 | 30.31 | 38.47 | 30.78 | 39.24 | 31.39 | 39.62 | 31.70 | 40.01 | 32.01 | 38.47 | 30.78 |
| (Parteck M100) | | | | | | | | | | | | |
| colloidal silicon | 2.50 | 2.00 | 2.50 | 2.00 | 2.50 | 2.00 | 2.50 | 2.00 | 2.50 | 2.00 | 2.50 | 2.00 |
| dioxide | | | | | | | | | | | | |
| (Aerosil 200) | | | | | | | | | | | | |
| Crospovidone | 7.50 | 6.00 | 7.50 | 6.00 | 7.50 | 6.00 | 7.50 | 6.00 | 7.50 | 6.00 | 7.50 | 6.00 |
| Strawberry | 0.13 | 0.10 | 0.13 | 0.10 | 0.13 | 0.10 | 0.13 | 0.10 | 0.13 | 0.10 | 0.13 | 0.10 |
| Flavor | | | | | | | | | | | | |
| Sodium stearyl | 3.75 | 3.00 | 3.75 | 3.00 | 3.75 | 3.00 | 3.75 | 3.00 | 3.75 | 3.00 | 3.75 | 3.00 |
| Fumarate | | | | | | | | | | | | |
| TOTAL | 125.00 | 100.00 | 125.00 | 100.00 | 125.00 | 100.00 | 125.00 | 100.00 | 125.00 | 100.00 | 125.00 | 100.00 |

The tablets were dissolved mixed at 5″0 rpm in 500 ml of HCL (pH 4.5). Three measurements were effected. The average of measurements are disclosed in Table 2.

The tablets were manufactured containing an increasing amount of the acidifier, tartaric acid. The dissolution data clearly showed that a tablet without acidifier resulted in a slower and incomplete release of Risdiplam in comparison to tablets containing tartaric acid. The benefit of the acidifier was already present at the lowest tartaric acid concentration. Increasing the percentage of acidifier in the formulation led to faster dissolution rate, with formulation 6 dissolving the most quickly amongst all tested formulations.

TABLE 2
Dissolution rate of tablets comprising Tartaric acid
(% of risdiplam in solution)

| | Sampling time [min] | | | | | |
|---|---|---|---|---|---|---|
| Formulation # | 3 | 6 | 9 | 15 | 30 | 45 |
| Formulation 1 | 14 | 31 | 39 | 49 | 57 | 83 |
| Formulation 6 | 59 | 82 | 84 | 88 | 93 | 97 |
| Formulation 5 | 18 | 70 | 86 | 90 | 93 | 95 |
| Formulation 4 | 16 | 74 | 85 | 89 | 93 | 96 |
| Formulation 3 | 25 | 69 | 77 | 82 | 88 | 93 |
| Formulation 2 | 14 | 63 | 78 | 84 | 88 | 92 |

### Example 2: Influence of Presence of Iron in the Coating and on the Stability of the Drug Product

The two tablets formulation 7 and Formulation 8 as described in Table 3 were made according to the process described below.

The tablets were produced on 130 g lab scale by a subsequent manual sieving step with a 0.9 mm screen, followed by tumble blending for 3 minutes. The lubricant was sieved through a 0.5 mm screen and pre-blended for 1 minute with a 10^{th} of the internal phase by using a tumble blender. The pre-blend and the rest of the internal phase was blended with a tumble blender for 3 minutes. The final blend was compressed to tablets by using a Styl'one single punch press.

TABLE 3
Composition of tablets for example 2

| | Formulation 7 | | Formulation 8 | |
|---|---|---|---|---|
| | mg | % | mg | % |
| Risdiplam | 5.00 | 3.85 | 5.00 | 3.85 |
| Parteck M100 | 76.70 | 59.00 | 76.70 | 59.00 |
| Corn Starch | 15.37 | 11.82 | 15.37 | 11.82 |
| Tartaric Acid | 7.53 | 5.79 | 7.53 | 5.79 |
| colloidal silicon | 2.50 | 1.92 | 2.50 | 1.92 |
| dioxide (Aerosil 200) | | | | |
| Croscarmellose-Sodium | 3.75 | 2.89 | 3.75 | 2.89 |
| Strawberry flavor PHS-180152 | 9.40 | 7.23 | 9.40 | 7.23 |
| Sucralose | 1.00 | 0.77 | 1.00 | 0.77 |
| Sodium stearyl Fumarate | 3.75 | 2.89 | 3.75 | 2.89 |
| Opadry II brown 85F26792 | — | — | 5.00 | 3.85 |
| (PVA based) ^{a} | | | | |
| Opadry II white 85F18422 | 5.00 | 3.85 | — | — |
| (PVA based) ^{b} | | | | |
| TOTAL | 130.00 | 100.00 | 130.00 | 100.00 |

^{a} comprises iron oxide
^{b} iron oxide free

Two tablet compositions (Formulation 7 & Formulation 8) were manufactured and stored for two weeks at 70° C./5% Relative Humidity (RH) or 70° C./75% RH respectively. Formulation 7 contains a coating free of iron oxide, whereas Formulation 8 contains an iron oxide coating. Initially (t0) and after two weeks storage, the Risdiplam concentration was determined. The results are shown in Table 4.

TABLE 4
Stability of the formulation 7 and 8 tablets

| | | API | Total |
|---|---|---|---|
| | | concentration | Degradation |
| Formulation # | Storage condition | [%] | [%]^{a} |
| Formulation 7 | t₀ | 96.0 | ≤0.10 |
| | 2 weeks, 70° C. 5% RH | 92.5 | 0.41 |
| | 2 weeks, 70° C. 75% RH | 60.3 | 14.30 |
| Formulation 8 | t₀ | 93.2 | ≤0.10 |
| | 2 weeks, 70° C. 5% RH | 90.6 | 0.45 |
| | 2 weeks, 70° C. 75% RH | 3.1 | 15.30 |

^{a}The total degradation corresponds to the degradation products known at the time of the analysis.

The experiment clearly showed, that decomposition is promoted at elevated humidity and especially in presence of iron oxide, where the Risiplam concentration was remarkable reduced over time (Formulation 8) in comparison to the tablet without iron oxide. Formulation 7, without iron oxide, demonstrated that storage at 2 weeks at elevated humidity (75% RH) resulted in over 30% more Risdiplam degradation compared to low humidity (5% RH). The presence of iron oxide (Formulation 8) at the same conditions resulted in a surprisingly increased degradation over 85% at elevated humidity, compared to low humidity. Accordingly, the presence of iron oxide in a tablet formulation of Risdiplam causes significant instability of the API. Formulation 8 turned deep black after two weeks storage at 70° C./75% RH as shown in FIG. 7. Based on Table 4, less than 5.0% of risdiplam could be recovered from the formulation 8, the formulation coated with an iron oxide based coating The decomposition comprises the following degradation products:
a) 1-(2,8-dimethylimidazo[1,2-b]pyridazin-6-yl)ethanone:

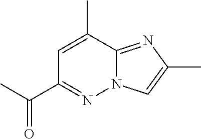

b) 7-(2-aminoethylamino)-2-(2,8-dimethylimidazo[1,2-b]pyridazin-6-yl)pyrido[1,2-a]pyrimidin-4-one:

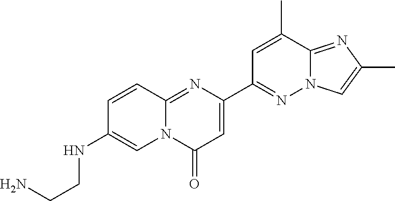 [SMILES: Cc1cn2nc(-c3cc(=O)n4cc(NCCN)ccc4n3)cc(C)c2n1]

c) 2-(2,8-dimethylimidazo[1,2-b]pyridazin-6-yl)-7-hydroxy-pyrido[1,2-a]pyrimidin-4-one:

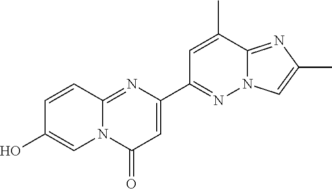 [SMILES: Cc1cn2nc(-c3cc(=O)n4cc(O)ccc4n3)cc(C)c2n1]

### Example 3: Impact of the Filler Composition on the Manufacturing Process and Tablet Core Performance

A series of prototype placebo tablets were manufactured containing different filler combinations in order to identify a composition which promotes good flowability of the powder blend, good tablet compression performance and provides good tablet properties with respect to hardness, abrasion and disintegration time. The tablets were compressed with an increasing compression force in order to assess the compressibility and resulting tablet properties for a given composition. Besides the fillers, other excipients were not varied as shown in Table 5. The tablets were made as described below. The physical characteristics of each tablet were assessed and are summarized in Table 6.

The tablets were produced on 1.5 kg lab scale by a subsequent manual sieving step with a 1.2 mm screen, followed by tumble blending for 5 min. The lubricant was sieved through a 0.5 mm screen and pre-blended for 1 minute with a 10^{th} of the internal phase by using a tumble blender. The pre-blend and the rest of the internal phase was blended with a tumble blender for 5 minutes. The final blend was compressed to tablets by using a Korsch XL-100 rotary press. The main compression force was varied between 1.8-4.6 kN in order to investigate the compaction profile.

TABLE 5
Composition of placebo tablets.

| | Formulation 9 | | Formulation 10 | | Formulation 11 | |
|---|---|---|---|---|---|---|
| | [mg] | [%] | [mg] | [%] | [mg] | [%] |
| API | 0.00 | — | 0.00 | — | 0.00 | — |
| Isomalt | 0.00 | — | 0.00 | — | 37.50 | 30.00 |
| Tartaric Acid | 7.53 | 6.02 | 7.53 | 6.02 | 7.53 | 6.02 |
| fine powder | | | | | | |
| Micro- | 65.10 | 52.08 | 65.10 | 52.08 | 65.10 | 52.08 |
| crystalline | | | | | | |
| Cellulose | | | | | | |
| (MCC | | | | | | |
| PH102) | | | | | | |
| Lactose Fast | 37.50 | 30.00 | — | — | — | — |
| Flo 316 | | | | | | |
| Mannitol | — | — | 37.50 | 30.00 | — | — |
| (Parteck | | | | | | |
| M100 ®) | | | | | | |
| colloidal | 2.50 | 2.00 | 2.50 | 2.00 | 2.50 | 2.00 |
| silicon | | | | | | |
| dioxide | | | | | | |
| (Aerosil | | | | | | |
| 200 ® | | | | | | |
| Cros- | 7.50 | 6.00 | 7.50 | 6.00 | 7.50 | 6.00 |
| povidone | | | | | | |
| Strawberry | 0.13 | 0.10 | 0.13 | 0.10 | 0.13 | 0.10 |
| Flavor | | | | | | |
| PHS-180152 | | | | | | |
| Sucralose | 1.00 | 0.80 | 1.00 | 0.80 | 1.00 | 0.80 |
| Sodium | 3.75 | 3.00 | 3.75 | 3.00 | 3.75 | 3.00 |
| Stearyl | | | | | | |
| Fumarate | | | | | | |
| TOTAL | 125.00 | 100.00 | 125.00 | 100.00 | 125.00 | 100.00 |

TABLE 6
Impact of the filler composition

| | | | Compression | | Disintegration | |
|---|---|---|---|---|---|---|
| | Filler | FFC | force | Hardness | time | Abrasion |
| Batch No. | combination | (@1000 Pa) | [kN] | [N] | [min:sec] | [%] |
| Formulation 9 | Microcrystalline | 8.25 | 1.8 | 46 | 00:13 | n/a |
| | Cellulose/ | | 2.2 | 56 | 00:22 | n/a |
| | Lactose | | 2.5 | 68 | 00:29 | n/a |
| | 63:37 ratio | | 3.2 | 80 | 01:02 | 0.29 |
| | | | 4.6 | 109 | 02:37 | n/a |
| Formulation 10 | Microcrystalline | 7.13 | 1.9 | 45 | 00:10 | n/a |
| | Cellulose/ | | 2.6 | 72 | 00:20 | n/a |
| | Mannitol | | 3.8 | 90 | 01:12 | 0.11 |
| | 63:37 ratio | | | | | |
| Formulation 11 | Microcrystalline | n/a | 1.8 | 41 | 00:30 | n/a |
| | Cellulose/ | | 2.1 | 43 | 00:46 | 0.51 |
| | Isomalt | | 2.5 | 62 | 02:34 | n/a |
| | 63:37 ratio | | 3.4 | 90 | 06:58 | n/a |

It was surprisingly found that formulation 10 was the best composition as it achieved a sufficient flow and the tablet core abrasion was low, signaling sufficient robustness for further processing (coating, packaging and transport) by still maintaining a fast disintegration time.

### Example 4: Impact of Level of Disintegrant on the Disintegration Time

The tablets mentioned in Table 7 were made according to the described process mentioned herein. The impact of different coating types on the dissolution of the two formulations was then evaluated, as summarized in Table 8.

The tablets were produced on 12.4 kg pilot scale by a subsequent sieving step with a ServoWitt conical mill with a 1.5 mm screen, followed by tumble blending (Servolift) for 7 minutes at 15 rpm. The lubricant was manually sieved through a 0.5 mm screen and added to the internal phase by. The final blend was blended with a tumble blender (Servolift) for 5 minutes at 15 rpm. The final blend was compressed to tablets by using a Fette 1090i rotary press and a target main compression force between 3-5 kN. Aqueous film-coating was performed with a Glatt drum coater.

TABLE 7
Composition of tablets used in example 4.

| | Formulation 12 | | Formulation 13 | | Placebo | |
|---|---|---|---|---|---|---|
| | [mg] | [%] | [mg] | [%] | [mg] | [%] |
| Risdiplam | 5.00 | 4.00 | 5.00 | 4.00 | 0.00 | 0.00 |
| Tartaric Acid fine powder | 7.53 | 6.02 | 3.00 | 2.40 | 7.53 | 6.02 |
| Microcrystalline Cellulose | 60.10 | 48.08 | 64.63 | 51.70 | 37.50 | 30.00 |
| (MCC PH102) | | | | | | |
| Mannitol | 37.50 | 30.00 | 37.50 | 30.00 | 65.10 | 52.08 |
| (Parteck M100) | | | | | | |
| colloidal silicon dioxide | 2.50 | 2.00 | 2.50 | 2.00 | 2.50 | 2.00 |
| (Aerosil 200) | | | | | | |
| Crospovidone | 7.50 | 6.00 | 7.50 | 6.00 | 7.50 | 6.00 |
| Strawberry flavor | 0.13 | 0.10 | 0.13 | 0.10 | 0.13 | 0.10 |
| Sucralose | 1.00 | 0.80 | 1.00 | 0.80 | 1.00 | 0.80 |
| Sodium Stearyl Fumarate | 3.75 | 3.00 | 3.75 | 3.00 | 3.75 | 3.00 |
| TOTAL | 125.00 | 100.00 | 125.00 | 100.00 | 125.00 | 100.00 |

TABLE 8
Coating effect on disintegration of Risdiplam

| | | Coating | Coating | Average | Minimum | Maximum |
|---|---|---|---|---|---|---|
| | | amount | amount | Disintegration | Disintegration | Disintegration |
| Formulation # | Coating type | [mg] | [%] | [min:sec] | [min:sec] | [min:sec] |
| Formulation 12 | Opadry II | 2.80 | 2.20 | 02:32 | 02:00 | 02:58 |
| | white | 4.25 | 3.40 | 02:46 | 02:24 | 03:04 |
| | 85F18422ª | 5.73 | 4.60 | 03:15 | 02:47 | 03:43 |
| | (PVA) | | | | | |
| Formulation 12 | Opadry II | 2.80 | 2.20 | 03:12 | 02:20 | 03:46 |
| | yellow | | | | | |
| | 85F220022^{b} | | | | | |
| | (PVA) | | | | | |
| Formulation 12 | Opadry II | 4.10 | 3.30 | 02:54 | 02:25 | 03:29 |
| | yellow | | | | | |
| | 85F220022^{b} | | | | | |
| | (PVA) | | | | | |
| Formulation 13 | Opadry II | 2.50 | 2.00 | 01:37 | 01:14 | 02:12 |
| | yellow | | | | | |
| | 85F220022^{b} | | | | | |
| | (PVA) | 4.10 | 3.30 | 01:56 | 01:30 | 02:24 |
| Formulation 13 | Opadry II | 5.50 | 4.40 | 01:46 | 01:33 | 01:57 |
| | white85F18422 | 6.90 | 5.50 | 02:02 | 01:52 | 02:24 |
| | (PVA)^{c} | 8.40 | 6.70 | 02:23 | 01:58 | 02:40 |

^{a}1.000 mg polyvinylalcohol, 0.625 mg titanium dioxide, 0.505 mg Polyethylene Glycol 3350 and 0.370 mg Talcum
^{b}1.000 mg polyvinylalcohol, 0.608 mg titanium dioxide, 0.505 mg Polyethylene Glycol 3350, 0.370 mg Talcum and 0.017 mg yellow iron oxide
^{c}polyvinyl alcohol, titanium dioxide, Polyethylene Glycol, talc

For batch Formulation 12 (high tartaric acid), increase of coating amount resulted in remarkable increase of the disintegration time. Already at low level, the target specification (<3 minutes) was exceeded. Batch Formulation 13 did not show such a pronounced effect and disintegration time remained below 3 minutes.

Batch Formulation 12 without coating was used to perform an accelerated stability assessment. The scale-up batch was incubated openly for two weeks at 70° C./75% RH and 70° C./5% RH.

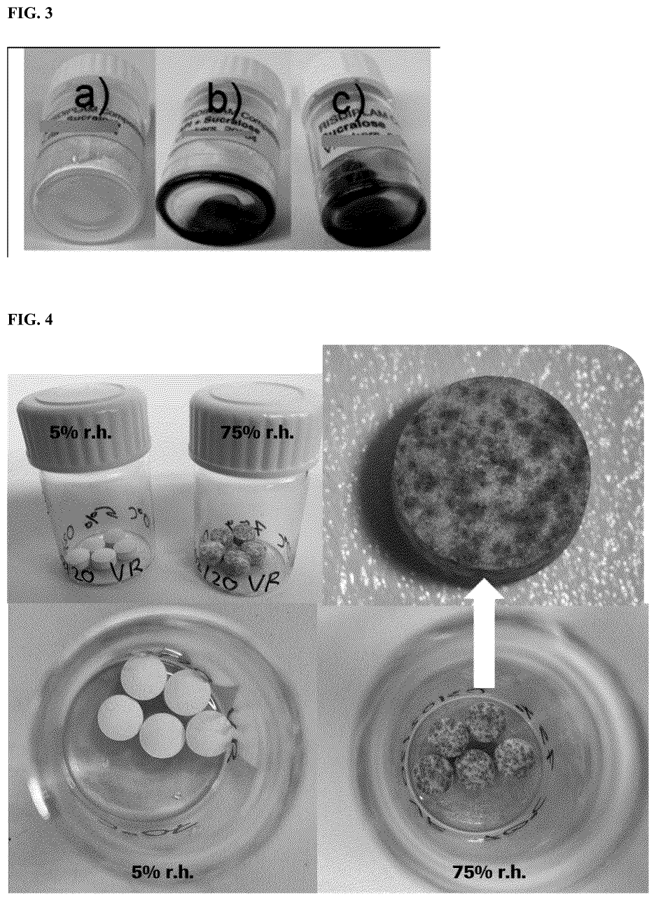

When exposed to high humidity (75% RH), a dramatic change of color was observed (see FIG. 4) and intense discoloration was accompanied by a strong decrease of the risdiplam content. Discoloration was directly linked to Risdiplam degradation, as placebo tablets did not show any change of color.

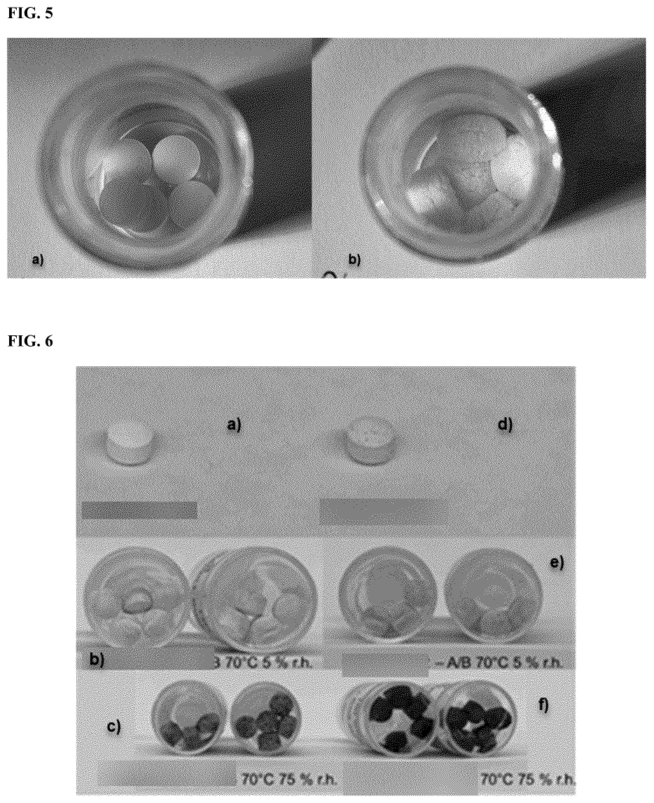

The discoloring can only be attributed to risdiplam, as placebo tablets (“Placebo”) were also incubated under the same conditions without coating, 70° C./5% RH for 2 weeks (FIG. 5a) and 70° C./75% RH for two weeks (FIG. 5b). No color change was observed. A swelling and increased porosity were highly evident in the placebo tablets stored under high humidity conditions. The placebo tablets stored at low humidity were not affected at all.

### Example 6: Formulation A

A pharmaceutical composition comprising risdiplam was prepared as mentioned below.

TABLE 9
Composition of tablet formulation (A):

| | Target | % w/w | |
|---|---|---|---|
| | amount per | Tablet | % w/w |
| | dosage unit | core | Total |
| Component | (mg) | weight | Weight |
| Risdiplam | 5.000 | 4.00% | 3.85% |
| L-Tartaric Acid (Powder) | 3.000 | 2.40% | 2.31% |
| Mannitol | 39.620 | 31.70% | 30.48% |
| Microcrystalline Cellulose | 63.505 | 50.80% | 48.85% |
| colloidal silicon dioxide | 2.500 | 2.00% | 1.92% |
| Crospovidone | 7.500 | 6.00% | 5.77% |
| Strawberry flavor (PHS-180152) | 0.125 | 0.10% | 0.10% |
| Sodium stearyl Fumarate | 3.750 | 3.00% | 2.88% |
| Subtotal Weight (Tablet Core) | 125.000 | | |
| Film Coat | | | |
| OPADRY II white 85F18422^{a} | 2.500 | | 1.92% |
| OPADRY II yellow 85F220022^{b} | 2.500 | | 1.92% |
| Total Weight (Film-coated Tablet) | 130.000 | | |

^{a}1.000 mg polyvinylalcohol, 0.625 mg titanium dioxide, 0.505 mg Polyethylene Glycol 3350 and 0.370 mg Talcum
^{b}1.000 mg polyvinylalcohol, 0.608 mg titanium dioxide, 0.505 mg Polyethylene Glycol 3350, 0.370 mg Talcum and 0.017 mg yellow iron oxide

A 14.4 kg batch was produced. Risdiplam, L-tartaric acid, mannitol, microcrystalline cellulose, colloidal silicon dioxide, crospovidone and strawberry flavor were weighed and transferred in a suitable metal drum and mixed for 7 min at a speed of 15 rpm. The mixture was then put through a 1.5 mm sieve manually in a container (A).

Sodium stearyl fumarate was manually sieved through a 0.5 mm sieve and added to the powder mixture of container (A). This is then blended for 7 min at 15 upm.

Tableting was carried out using tablet tooling on a standard manufacturing tablet machine and a target main compression force between 4-4.65 kN. Aqueous film-coating was performed with a Glatt drum coater.

### Example 7: Formulation B

A pharmaceutical composition comprising risdiplam was prepared as mentioned below.

TABLE 10
Composition of tablet formulation (B)

| | Target | % w/w | |
|---|---|---|---|
| | amount per | Tablet | % w/w |
| | dosage unit | core | Total |
| Component | (mg) | weight | Weight |
| Risdiplam | 5.000 | 4.00% | 3.85% |
| Mannitol | 40.775 | 32.62% | 31.37% |
| Microcrystalline Cellulose | 65.350 | 52.28% | 50.27% |
| colloidal silicon dioxide | 2.500 | 2.00% | 1.92% |
| Crospovidone | 7.500 | 6.00% | 5.77% |
| Strawberry Flavor (PHS-180152) | 0.125 | 0.10% | 0.10% |
| Sodium stearyl Fumarate | 3.750 | 3.00% | 2.88% |
| Subtotal Weight (Tablet Core) | 125.000 | | |
| Film Coat | | | |
| OPADRY II white 85F18422^{a} | 2.500 | | 1.92% |
| OPADRY II yellow 85F220022^{b} | 2.500 | | 1.92% |
| Total Weight (Film-coated Tablet) | 130.000 | | |

^{a}1.000 mg polyvinylalcohol, 0.625 mg titanium dioxide, 0.505 mg Polyethylene Glycol 3350 and 0.370 mg Talcum
^{b}1.000 mg polyvinylalcohol, 0.608 mg titanium dioxide, 0.505 mg Polyethylene Glycol 3350, 0.370 mg Talcum and 0.017 mg yellow iron oxide

A 14.4 kg batch was produced. Risdiplam, cellulose, L-tartaric acid, mannitol, Microcrystalline cellulose, colloidal silicon dioxide crospovidone and strawberry flavor were weighed and transferred in a suitable metal drum and mixed for 7 min at a speed of 15 rpm. The mixture was then put through a 1.5 mm sieve manually in a container (A).

Sodium stearyl fumarate was manually sieved through a 0.5 mm sieve and added to the powder mixture of container (A). This was then blended for 7 min at 15 rpm.

Tableting was carried out using tablet tooling on a standard manufacturing tablet machine and a target main compression force between 4-4.65 kN. Aqueous film-coating was performed with a Glatt drum coater.

### Example 8: Sodium Croscarmellose Vs Crospovidone

60 mg of Tartaric Acid were dissolved in 100 ml tap water (0.6 mg/ml) at room temperature and protected from light. A representative amount of Sodium Croscarmellose or Crospovidone was added and dispersed. After that, 100 mg of risdiplam were added, the suspension stirred for 30 minutes und the concentration analyzed by UPLC (see Table 11).

TABLE 11
Impact of Sodium Croscarmellose and
Crospovidone on Risdiplam solubility

| | | Concentration [mg/ml] |
|---|---|---|
| Excipient (mg) | pH | (Nominal 1 mg/ml) |
| Sodium Croscarmellose (75) | 4.1 | 0.28 |
| Crospovidone (75) | 4.1 | 0.97 |

### Example 9 Impact of Sucralose on Risdiplam

The impact of Sucralose on Risdiplam stability was evaluated. The results are shown in FIG. 3. Vial (a) contained only risdiplam and is shown at time 0; vial (b) is 25 mg risdiplam and 475 mg Sucralose after 15 days at 70° C./75% Relative Humidity; and vial (c) is Sucralose without Risdiplam after 15 days at 70° C./75% Relative Humidity. Sucralose was found to cause discoloration and promote the decomposition of risdiplam.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1-51. (canceled)

52. A pharmaceutical composition, comprising:
(a) a compound of formula (I): 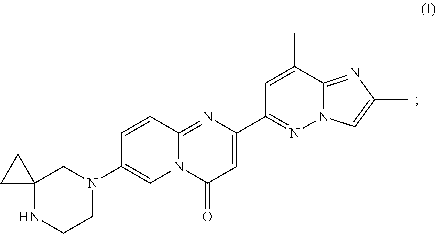 [SMILES: Cc1cn2nc(-c3cc(=O)n4cc(N5CCNC6(CC6)C5)ccc4n3)cc(C)c2n1]
(b) crospovidone; and
(c) an acidifier selected from the group consisting of tartaric acid, citric acid, and malic acid;
wherein the pharmaceutical composition is in the form of a tablet.

53. The pharmaceutical composition of claim 52, wherein the tablet comprises:
i) a tablet Kernel comprising:
(a) the compound of formula (I);
(b) crospovidone; and
(c) an acidifier selected from the group consisting of tartaric acid, citric acid, and malic acid; and
ii) at least one film coating.

54. The pharmaceutical composition of claim 53, wherein the at least one film coating comprises a first film coating, wherein the first film coating directly contacts the tablet Kernel.

55. The pharmaceutical composition of claim 54, wherein the first film coating comprises polyvinyl alcohol, and comprises less than 0.001% by weight of iron oxide.

56. The pharmaceutical composition of claim 53, wherein the tablet comprises a second film coating, wherein the second film coating coats the first film coating.

57. The pharmaceutical composition of claim 56, wherein the second film coating comprises iron oxide.

58. The pharmaceutical composition of claim 52, wherein the pharmaceutical composition further comprises a filler selected from the group consisting of microcrystalline cellulose, cellulose powder, isomalt, lactose, maltodextrin, mannitol, dibasic calcium phosphate, sorbitol, starch, calcium sulfate, and any combinations thereof.

59. The pharmaceutical composition of claim 52, wherein the pharmaceutical composition further comprises:
a filler selected from the group consisting of lactose, starch, maltodextrin, isomalt, microcrystalline cellulose, mannitol, sorbitol, sucrose, dextrose, dibasic calcium phosphate, calcium sulfate, and any combinations thereof,
a lubricant selected from sodium stearyl fumarate and magnesium stearate; and
a glidant.

60. The pharmaceutical composition of claim 52, wherein the pharmaceutical composition further comprises:
a filler, wherein the filler is microcrystalline cellulose and mannitol;
a lubricant, wherein the lubricant is sodium stearyl fumarate; and
a glidant, wherein the glidant is colloidal silicon dioxide.

61. The pharmaceutical composition of claim 52, wherein the pharmaceutical composition comprises less than 0.001% by weight disodium ethylenediaminetetraacetate.

62. The pharmaceutical composition of claim 52, wherein the pharmaceutical composition comprises less than 0.001% by weight of sucralose.

63. The pharmaceutical composition of claim 52, wherein the acidifier is tartaric acid, and the pharmaceutical composition comprises:
between 2% and 8% by weight of the compound of formula (I); and
between 1.0% and 4.0% by weight of tartaric acid.

64. The pharmaceutical composition of claim 63, wherein the pharmaceutical composition further comprises:
between 20% and 40% by weight of mannitol;
between 0.2% and 5.0% by weight of colloidal silicon dioxide;
between 0.2% and 5% by weight of sodium stearyl fumarate;
between 20% and 60% by weight of microcrystalline cellulose; and
between 4.0% and 8.0% by weight of crospovidone.

65. The pharmaceutical composition of claim 55, wherein:
the acidifier is tartaric acid;
the pharmaceutical composition comprises:
between 2% and 8% by weight of the compound of formula (I); and
between 1.0% and 4.0% by weight of tartaric acid;
less than 0.001% by weight disodium ethylenediaminetetraacetate; and
less than 0.001% by weight sucralose.

66. The pharmaceutical composition of claim 65, wherein the tablet comprises a second film coating, wherein the second film coating comprises iron oxide and coats the first film coating.

67. The pharmaceutical composition of claim 53, wherein the acidifier is tartaric acid, and the pharmaceutical composition comprises less than 0.001% by weight disodium ethylenediaminetetraacetate; and less than 0.001% by weight sucralose.

68. The pharmaceutical composition of claim 52, comprising:
between 2% and 8% by weight of the compound of formula (I);
between 4.0% and 8.0% by weight of crospovidone; and
less than 0.001% by weight disodium ethylenediaminetetraacetate.

69. The pharmaceutical composition of claim 53, comprising:
between 2% and 8% by weight of the compound of formula (I);
between 4.0% and 8.0% by weight of crospovidone; and
less than 0.001% by weight disodium ethylenediaminetetraacetate;
and wherein the first film coating comprises less than 0.001% by weight of iron oxide.

70. The pharmaceutical composition of claim 52, comprising:
(a) between 2% and 8% by weight the compound of formula (I);
(b) between 4.0% and 8.0% by weight of crospovidone;
(c) between 1.0% and 4.0% by weight of an acidifier selected from the group consisting of tartaric acid, citric acid, and malic acid;
(d) between 70% and 90% by weight of a filler selected from the group consisting of microcrystalline cellulose, cellulose powder, isomalt, lactose, maltodextrin, mannitol, dibasic calcium phosphate, sorbitol, sucrose, dextrose, hydrolyzed starch, corn starch, pregelatinized starch, calcium sulfate, and any combinations thereof,
(e) between 2.0% and 4.0% by weight of a lubricant selected from the group consisting of sodium stearyl fumarate and magnesium stearate;
(f) between 1.0% and 3.0% by weight of a glidant, wherein the glidant is colloidal silicon dioxide; and
(g) less than 0.001% by weight of disodium ethylenediaminetetraacetate.

71. The pharmaceutical composition of claim 70, wherein the tablet comprises:
i) a tablet Kernel comprising the compound of formula (I), crospovidone, acidifier, filler, lubricant, and glidant; and
ii) at least one film coating, wherein the at least one film coating comprises a first film coating, wherein the first film coating directly contacts the tablet Kernel and comprises polyvinyl alcohol and less than 0.001% by weight of iron oxide.

72. The pharmaceutical composition of claim 52, wherein the pharmaceutical composition comprises:
i) a tablet Kernel comprising:
(a) between 2% and 8% by weight of the compound of formula (I);
(b) between 1.0% and 4.0% by weight of an acidifier selected from tartaric acid, citric acid, and malic acid;
(c) between 0.5% and 12.0% by weight of crospovidone;
(d) between 20% and 60% by weight of microcrystalline cellulose;
(e) between 20% and 40% by weight of mannitol;
(f) between 0.2% and 5% by weight of sodium stearyl fumarate; and
(g) between 0.2% and 5.0% by weight of colloidal silicon dioxide; and
ii) at least one film coating, wherein the at least one film coating comprises a first film coating, wherein the first film coating directly contacts the tablet Kernel and comprises less than 0.001% by weight of iron oxide.

73. The pharmaceutical composition of claim 72, wherein the acidifier is tartaric acid, and wherein the pharmaceutical composition comprises less than 0.001% by weight disodium ethylenediaminetetraacetate and less than 0.001% by weight sucralose.

74. The pharmaceutical composition of claim 73, wherein the tablet comprises a second film coating, wherein the second film coating comprises iron oxide and coats the first film coating.

75. The pharmaceutical composition of claim 52, comprising about 5 mg of the compound of formula (I).

76. The pharmaceutical composition of claim 55, comprising about 5 mg of the compound of formula (I).

77. The pharmaceutical composition of claim 63, comprising about 5 mg of the compound of formula (I).

78. The pharmaceutical composition of claim 65, comprising about 5 mg of the compound of formula (I).

79. The pharmaceutical composition of claim 73, comprising about 5 mg of the compound of formula (I).
